(19) United States
(12) Patent Application Publication
(10) Pub. No.: US 2024/0327571 A1
(43) Pub. Date: Oct. 3, 2024

(54) CROSSLINKED POLYMER ELASTOMERS, COMPOSITIONS, AND METHODS OF PREPARATION THEREOF

(71) Applicant: MOMENTIVE PERFORMANCE MATERIALS INC., Niskayuna, NY (US)

(72) Inventors: Alok SARKAR, Bangalore (IN); Pushpavathi J, Bangalore (IN); Debarshi DASGUPTA, Bangalore (IN); Ashitha KANDIKKAL, Bangalore (IN); Xu QIN, Northvale, NJ (US); Koushik MUKHERJEE, Bangalore (IN); Sigfredo GONZALEZ, Danbury, CT (US)

(21) Appl. No.: 18/618,718

(22) Filed: Mar. 27, 2024

Related U.S. Application Data
(60) Provisional application No. 63/492,305, filed on Mar. 27, 2023.

(51) Int. Cl. C08G 63/16; C08G 63/81

(52) U.S. Cl. CPC C08G 63/16; C08G 63/81; C08J 2367/02

(57) ABSTRACT

The present disclosure provides polyester elastomers, compositions, and methods of preparing such elastomers and compositions. The polyester elastomers in the present disclosure are prepared by reacting at least one activated polycarboxylic acid with at least one polyol. The crosslinked polyester elastomer compositions can be formulated into various personal care formulations.

### FIELD

The present disclosure provides polyester elastomers, compositions, and methods of preparing such elastomers and compositions. These elastomers are prepared by reacting at least one activated polycarboxylic acid and at least one polyol. Further, these elastomers can be converted to elastomer powders and gels. These elastomers and elastomer gels can be biodegradable and produced from biorenewable raw materials. Further, these elastomer gels provide advantageous properties when combined with various personal care products.

### BACKGROUND

Crosslinked polymers are added to manipulate the sensory, texture, rheology, and optical performance of a variety of cosmetic products. Silicone elastomers are particularly important because they can form elastic particles of three-dimensional polymeric dimethicone and provide a beneficial sensory, texture, and optical effect to cosmetic products. However, traditional silicone elastomers have limited versatility in terms of compatibility with polar solvents such as hydrocarbon oils, plant-based oils, glycerin, and water. Therefore, although the performance of silicone elastomers is unparalleled, there is a demand for alternatives to silicone elastomers. In particular, there is a demand in the marketplace for non-silicone based elastomer materials.

Many polyesters and polyurethanes are known and listed in the EU public cosmetic ingredients database. Some of these ingredients are synthetically made pre-polymers that are crosslinked with di- or tri-functional amine-based chain extenders. However, amine-based building blocks are not desirable for the development of cosmetic raw material due to their high odor and yellowing issues. Micronized polyurethanes particles based on synthetic feedstocks are available in market. These materials, while exhibiting excellent tactile and soft focus (optical) properties, do not have elastic properties analogous to silicone elastomers. U.S. Patent Appl. No. 20210059924A1 discloses alternative bio-based polyurethane elastomers developed based on crosslinking linear copolymers of aliphatic fatty acid dimers and diols with bio-based aliphatic polyisocyanate. However, use of isocyanates is often undesirable for the development of a cosmetic raw material due to the potential risk of isocyanate exposure in the production setup (Environ. Health Perspect. 2007, 115(3): 328-335). U.S. Patent Appl. No. 20220195178A1 discloses polyester-based elastomer compositions prepared by direct esterification reaction between a fatty acid and a fatty alcohol with multiple functionalities. However, crosslinking highly branched fatty acids using conventional esterification methods is extremely slow, reversible, and incomplete. Polyester elastomers prepared by this method often contains unreacted fatty acid and fatty alcohol monomers and oligomers which negatively impact the elastic properties of the elastomers. Therefore, there still exists a need to develop high performing natural elastomer compositions to meet the sensory profile of a silicone gel.

Crosslinked polyester elastomers known today are produced by traditional Fisher-Speier esterification in which carboxylic acids are directly condensed with an alcohol under strong acid Brønsted catalysis. The reactions are reversible and require continuous removal of water by a high temperature distillation process. Despite being very effective esterification catalysts, strong Brønsted acids also give rise to unwanted side reactions such as the dehydrative etherification of alcohols (ChemCatChem 2020, 12, 5229-5235). Although the direct, uncatalyzed transformation of a carboxylic acid and an alcohol to an ester is possible, it requires temperatures up to 250° C. to achieve full conversion under equilibrium conditions (J. Otera and J. Nishikido, Esterification: Methods, Reactions, and Applications, Wiley-VCH Verlag GmbH & Co., Weinheim, 2010). Therefore, crosslinked polyester elastomers produced today contain appreciable amounts of undesired side products which often have a negative impact on their performance. The present invention provides a method of preparing crosslinked polyester compositions comprising the reaction product of an activated poly fatty acid—where the activated poly fatty acid is generated in-situ or ex-situ using a suitable activating agent, a polyol, and a suitable solvent in the presence of a catalyst. The activated acid groups described herein undergo an esterification reaction with polyols to form carbon dioxide instead of water as byproduct. The spontaneous removal of carbon dioxide gas from the reaction mixture drives the esterification reaction to completion affording high purity elastomer with no or a negligible amount of unreacted starting monomers. These elastomers deliver enhanced rheological performance compared to analogous elastomers disclosed in the art.

In an aspect, this invention provides a personal care composition containing polyester elastomers.

Polyesters are a class of compounds that contain ester functional groups in their polymer chain. The ester groups can be hydrolyzed when treated with certain biological catalysts or certain mixed cultures of microorganisms—which make a large number of polyesters biodegradable. There is a significantly growing interest in recent years to design and develop biobased polyesters from renewable resources to use as emollient, emulsifier, film former, or other functional ingredient for personal care formulations. See for example, U.S. Pat. Nos. 8,414,906; 9,334,358; 6,540,987; 7,820,758. However, no polyester elastomer or gel has yet been reported that provides multiple benefits to consumers as a substitute for silicone gel.

### SUMMARY

The present invention discloses high purity crosslinked polyester elastomers comprising the reaction product of an activated poly fatty acid—where the activated poly fatty acid is generated in-situ or ex-situ using a suitable activating agent, a polyol, and a suitable solvent in presence of a catalyst. The activated acid groups disclosed here undergo crosslinking reaction with polyols to form carbon dioxide (gas) instead of water as byproduct. The spontaneous removal of carbon dioxide from the reaction mixture drives the esterification reaction in the forward direction toward completion affording a high purity elastomer with no or a negligible amount of unreacted starting monomers. These elastomers are expected to deliver superior performance benefits such as improved sensory, structuring, and rheological performance compared to their analogous elastomers disclosed previously. In an aspect, this invention relates to a personal care composition containing such high purity polyester elastomers.

In an aspect, the present disclosure provides an elastomer comprising the reaction product of
(i) at least one activated polycarboxylic acid; and
(ii) at least one polyol;
wherein (a) the at least one polycarboxylic acid and at least one polyol have a total of at least five carboxyl and hydroxyl functional groups; and (b) there are at least three carboxyl or hydroxyl functional groups on at least one activated polycarboxylic acid or at least one polyol.

In an aspect, the present disclosure provides an elastomer prepared by reacting:
(i) at least one polycarboxylic acid of formula (III)

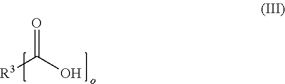

wherein
R³ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
o is an integer from 2 to 10;
(ii) at least one activating agent of formula (IV)

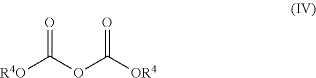

wherein
R⁴ represents a straight-chain or branched alkyl radical with C₁-C₆₀ atoms; and
(iii) at least one polyol of formula (II)

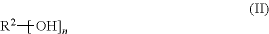

wherein
R² is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
n is an integer from 2 to 10.

In an aspect, the present invention provides a method of preparing an elastomer composition comprising reacting:
(i) at least one activated polycarboxylic acid; and
(ii) at least one polyol; in the presence of at least one solvent thereby forming a crosslinked polymer structure;
wherein (a) at least one activated polycarboxylic acid and at least one polyol have a total of at least five carboxyl and hydroxyl functional groups; and (b) there are at least three carboxyl or hydroxyl functional groups on at least one polycarboxylic acid or at least one polyol.

In an aspect, the present disclosure provides a method of preparing an elastomer comprising reacting:
(i) at least one activated polycarboxylic acid; and
(ii) at least one polyol; in the presence of at least one solvent thereby forming a crosslinked polymer structure;
(iii) combining the crosslinked polymer structure with a second solvent thereby forming a swollen crosslinked polymer structure; and
(iv) subjecting the swollen crosslinked polymer structure to shear force thereby forming a uniform polyester elastomer gel;
wherein (a) at least one activated polycarboxylic acid and at least one polyol have a total of at least five carboxyl and hydroxyl functional groups; and (b) there are at least three carboxyl or hydroxyl functional groups on at least one polycarboxylic acid or at least one polyol.

In an aspect, the present disclosure provides for use of a gel prepared from an elastomer described herein as a personal care formulation.

### DETAILED DESCRIPTION

### I. Definitions

Unless otherwise indicated, any atom with unsatisfied valences is assumed to have hydrogen atoms sufficient to satisfy the valences.

It is to be noted that the term “a” or “an” entity refers to one or more of that entity; for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the region of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the following definitions shall apply unless otherwise indicated. For purposes of the present disclosure, the chemical elements are identified in accordance with the Periodic Table of the Elements, CAS version, and the Handbook of Chemistry and Physics, 75^{th} Ed. 1994. Additionally, general principles of organic chemistry are described in “Organic Chemistry,” Thomas Sorrell, University Science Books, Sausalito: 1999, and “March's Advanced Organic Chemistry,” 6^{th} Ed., Smith, M. B. and March, J., eds. John Wiley & Sons, New York: 2007, the entire contents of which are hereby incorporated by reference in their entireties.

The term “hydrocarbon”, as used herein by itself or as part of a group, refers to a straight- or branched-chain aliphatic series of one to two hundred carbon atoms, i.e., a C₁-C₂₀₀ hydrocarbon, or the number of carbon atoms designated, e.g., a C₁ hydrocarbon such as a methyl, a C₂ hydrocarbon such as ethyl, etc. In one embodiment, the hydrocarbon is a C₂-C₂₀₀ hydrocarbon group. In an embodiment, the hydrocarbon is a C₆-C₆₀ hydrocarbon group. In an embodiment, the hydrocarbon is a C₆-C₆₀ hydrocarbon group. In an embodiment, the hydrocarbon is a C₂-C₆₀ hydrocarbon group. In an embodiment, the hydrocarbon is a C₅-C₂₂ hydrocarbon group. Examples of hydrocarbon groups include butyl, octyl, decyl, lauryl, cetyl (palmityl), and stearyl.

The term “alkyl”, as used herein by itself or as part of a group, refers to a straight-or branched-chain aliphatic hydrocarbon containing one to two hundred carbon atoms, i.e., a C₂-C₂₀₀ alkyl, or the number of carbon atoms designated, e.g., a C₁ alkyl such as methyl, a C₂ alkyl such as ethyl, etc. In one embodiment, the alkyl is a C₂-C₂₀₀ alkyl group. In another embodiment, the alkyl is a C₆-C₆₀ alkyl group. In another embodiment, the alkyl is a C₂-C₆₀ alkyl group. In another embodiment, the alkyl is a C₅-C₂₂ alkyl group. Examples of alkyl groups include butyl, octyl, decyl, lauryl, cetyl (palmityl), and stearyl.

The term “alkene”, as used herein by itself or as part of a group, refers to an alkyl group containing one, two, three, or more carbon-to-carbon double bonds. In one embodiment, the alkene group is a C₂-C₂₀₀ alkylene group. In another embodiment, the alkene group is a C₆-C₆₀ alkene group. In another embodiment, the alkene group is a C₆-C₆₀ alkene group. In another embodiment, the alkene group is a C₂-C₆₀ alkene group. In another embodiment, the alkene group is a C₅-C₂₂ alkene group.

The term “alkyne”, as used herein by itself or as part of a group, refers to an alkyl group containing one, two, three, or more carbon-to-carbon triple bonds. In another embodiment, the alkyne is a C₂-C₂₀₀ alkyne group.

The term “cyclic”, as used herein by itself or as part of a group, refers to a stable cyclic compound containing three or more atoms. In an embodiment, the cyclic is a C₃-C₂₀₀ cyclic group. In an embodiment, the cyclic is a C₆-C₆₀ cyclic group. In an embodiment, the cyclic is a C₅-C₂₂ cyclic group. Examples of cyclic compounds include benzene, cyclopentane, and cyclohexane.

The term “heteroalkyl”, as used herein by itself or as part of a group, refers to a stable straight or branched chain alkyl radical containing two to two hundred carbon atoms and at least one heteroatoms, which can be the same or different, selected from O, N, or S, wherein the sulfur atom(s) can optionally be oxidized. The heteroatoms can be placed at any interior position of the heteroalkyl group or at a position at which the heteroalkyl group is attached to the remainder of the molecule. In an embodiment, the heteroalkyl is a C₆-C₆₀ heteroalkyl group. In an embodiment, the heteroalkyl is a C₂-C₆₀ heteroalkyl group. Examples of heteroalkyl compounds include succinyl, adipoyl, and sebacoyl.

The term “heteroalkene”, as used herein by itself or as part of a group, refers to a stable straight or branched chain alkene radical containing two to two hundred carbon atoms and at least one heteroatoms, which can be the same or different, selected from O, N, or S, wherein the sulfur atom(s) can optionally be oxidized. The heteroatoms can be placed at any interior position of the heteroalkyl group or at a position at which the heteroalkyl group is attached to the remainder of the molecule. In an embodiment, the heteroalkene is a C₆-C₆₀ heteroalkene group. In an embodiment, the heteroalkene is a C₂-C₆₀ heteroalkene group. Examples of heteroalkene compounds include oleoyl, ricinolyl, and linoleoyl.

The term “heteroalkyne”, as used herein by itself or as part of a group, refers to a stable straight or branched chain alkyne radical containing two to two hundred carbon atoms and at least one heteroatom, which can be the same or different, selected from O, N, or S, wherein the sulfur atom(s) can optionally be oxidized. The heteroatoms can be placed at any interior position of the heteroalkyl group or at a position at which the heteroalkyl group is attached to the remainder of the molecule.

The term “heterocyclic”, as used herein by itself or as part of a group, refers to a stable cyclic compound containing two or more carbons atoms and at least one heteroatom, which can be the same or different, selected from O, N, or S, wherein the sulfur atom(s) can optionally be oxidized. In an embodiment, the heterocyclic is a C₂-C₂₀₀ heterocyclic group. In an embodiment, the heterocyclic is a C₆-C₆₀ heterocyclic group. In an embodiment, the heterocyclic is a C₅-C₂₂ heterocyclic group. Examples of heterocyclic compounds include furan, oxolane, and thiophene.

The term “activated polycarboxylic acid”, as used herein or a part of a group, refers to a repeating unit of connected mixed anhydrides that are repeated an integer number of 2 to 10 times. The terminal end of the mixed anhydride is a hydrocarbon of length from C₁-C₆₀ carbon atoms. The nonrepeated end of the mixed anhydride is selected from the group consisting of a C₂-C₂₀₀ alkyl group, a C₂-C₂₀₀ heteroalkyl group, a C₂-C₂₀₀ alkene group, a C₂-C₂₀₀ heteroalkene group, a C₂-C₂₀₀ alkyne group, a C₂-C₂₀₀ heteroalkyne group, a C₃-C₂₀₀ cyclic group, or a C₂-C₂₀₀ heterocyclic group. The activated polycarboxylic acid can be generated through the reaction of a polycarboxylic acid and an activating agent.

As used herein, the term “olefin” refers to any species having at least one ethylenic double bond such as normal and branched chain aliphatic olefins, cycloaliphatic olefins, aryl substituted olefins, and the like. The olefin can comprise terminal double bond(s) (“terminal olefin”) and/or internal double bond(s) (“internal olefin”) and can be cyclic or acyclic, linear or branched, optionally substituted. The total number of carbon atoms can be from 1 to 100, or from to 40: the double bonds can be unsubstituted or mono-, bi-, tri- or tetrasubstituted.

As used herein, the term “polyolefin” refers to a homopolymer or copolymer of ethylene, propylene, butenes and other unsaturated aliphatic hydrocarbons, vinyl esters (e.g. vinyl acetate), or (meth)acrylics (e.g. butyl acrylate, acrylic acid). Generally, the polyolefin will be a polymer of ethylene, propylene or copolymer thereof, or a copolymer of ethylene or propylene with one or more C₄-C₁₂ α-olefin aliphatic comonomers.

Various aspects of the disclosure are described in greater detail below.

### II. Compositions

In one aspect, the present disclosure is directed to a crosslinked polyester elastomer comprising the reaction product of
at least one activated polycarboxylic acid; and
at least one polyol;
wherein (a) at least one activated polycarboxylic acid and at least one polyol have a total of at least five carboxyl and hydroxyl functional groups and (b) there are at least three carboxyl or hydroxyl functional groups on at least one polycarboxylic acid or at least one polyol.

### A. Components

### 1. Activated Polycarboxylic Acid

In an aspect, the at least one activated polycarboxylic acid is a compound of formula (I)

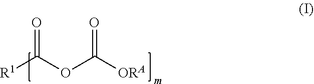

wherein
R¹ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group;
R^{A} is a C₁-C₆₀ monovalent hydrocarbon group; and
m is an integer from 2 to 10.

In an aspect, the compound is formula (I), wherein R¹ is C₆-C₆₀ alkyl group, C₆-C₆₀ heteroalkyl group, C₆-C₆₀ alkene group, C₆-C₆₀ heteroalkene group, C₆-C₆₀ cyclic group, or C₆-C₆₀ heterocyclic group; and m is an integer from 2 to 10.

In an aspect, the compound is formula (I), wherein m is an integer from 2 to 6. In an aspect, the compound is formula (I), wherein m is 2, 3, 4, 5, or 6.

In an aspect, at least one activated polycarboxylic acid is a compound generated in-situ or ex-situ by reacting a polycarboxylic acid of formula (III)

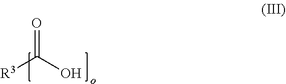

wherein
R³ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
o is an integer from 2 to 10;
with an activating agent.

In a further aspect, the compound is formula (III), wherein R³ is C₆-C₆₀ alkyl group, C₆-C₆₀ heteroalkyl group, C₆-C₆₀ alkene group, C₆-C₆₀ heteroalkene group, C₆-C₆₀ cyclic group, or C₆-C₆₀ heterocyclic group; and o is an integer from 2 to 10.

In an aspect, the compound is formula (III), wherein o is an integer from 2 to 6. In another aspect, the compound is formula (III), wherein o is 2, 3, 4, 5, or 6.

In an aspect, the polycarboxylic acid of formula (III) is selected from the group consisting of succinic acid, adipic acid, azelaic acid, sebacic acid, dodecanedioic acid, hexadecanedioic acid, C₂₁ dimer acid, C₃₆ dimer acid, hydrogenated C₃₆ dimer acid, aspartic acid, glutamic acid, tartaric acid, malic acid, and combinations thereof. In an aspect, the polycarboxylic acid is a dimer acid.

In an aspect, the at least one activated polycarboxylic acid is a mixture of activated polycarboxylic acids generated in-situ or ex-situ by reacting a dimer acid and an oleic acid with an activating agent.

In an aspect, at least one activated polycarboxylic acid is generated in-situ or ex-situ by reacting a polycarboxylic acid with an activating agent of formula (IV)

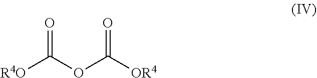

wherein
R⁴ represents a straight-chain or branched alkyl radical with C₁-C₆₀ atoms.

In an aspect, the activating agent is selected from the group consisting of dimethyl dicarbonate, diethyl dicarbonate, dipropyl dicarbonate, di-tertiary-butyl dicarbonate, and combinations thereof. In an aspect, the activating agent is di-tertiary-butyl dicarbonate.

In an aspect, the compound is formula (IV), wherein R⁴ is C₆-C₆₀ alkyl group, C₆-C₆₀ heteroalkyl group, C₆-C₆₀ alkene group, C₆-C₆₀ heteroalkene group, C₆-C₆₀ cyclic group, or C₆-C₆₀ heterocyclic group.

### 2. Polyol

In an aspect, at least one polyol is a compound of formula (II)

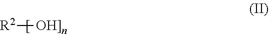

wherein
R² is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
n is an integer from 2 to 10.

In an aspect, the compound is formula (II), wherein R² is C₂-C₂₀₀ alkyl group, C₂-C₂₀₀ heteroalkyl group, C₂-C₂₀₀ alkene group, or C₂-C₂₀₀ heteroalkene group; and n is an integer from 2 to 10. In a further aspect, the compound is formula (II), wherein R² is C₂-C₆₀ alkyl group, C₂-C₆₀ heteroalkyl group, C₂-C₆₀ alkene group, or C₂-C₆₀ heteroalkene group; and n is an integer from 2 to 10.

In an aspect, the compound is formula (II), wherein n is an integer from 2 to 6. In an aspect, the compound is formula (II), wherein n is 2, 3, 4, 5, or 6.

In an aspect, the polyol is selected from the group consisting of glycerol, diglycerol, polyglycerol, sorbitol, castor oil, hydrogenated castor oil, sugar alcohol, monosaccharide, disaccharides, oligosaccharide, polysaccharides, tannin, gallic acid, gluconic acid, lactobionic acid, gluconolactone ethyleneglycol, 1,2-propanediol, 1,3-propanediol, 1,4-butanediol, 1,2-pentanediol, 1,3-pentanediol, 1,4-pentanediol, 1,5-pentanediol, 1,2-hexanediol, 1,5-hexanediol, 1,6-hexanediol, C₃₆ dimer diol, hydrogenated C₃₆ dimer diol, and combinations thereof. In an aspect, the polyol is hydrogenated castor oil. In an aspect, the polyol is diglycerol.

### B. Additional Components

In an aspect, the elastomer composition is prepared by reacting:
(i) at least one polycarboxylic acid of formula (III)

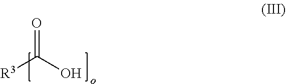

wherein
R³ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
o is an integer from 2 to 10;
(ii) at least one activating agent of formula (IV)

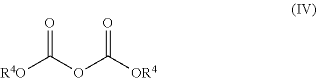

wherein
R⁴ represents a straight-chain or branched alkyl radical with C₁-C₆₀ atoms; and
(iii) at least one polyol of formula (II)

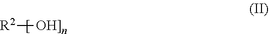

wherein
R² is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
n is an integer from 2 to 10.

### 1. Ratio

In an aspect, the molar ratio of total carboxyl functional groups (—COOH) to the activating agent is from about 1.5:1 to about 1:10. In an aspect, the molar ratio of total carboxyl functional groups to the activating agent is from about 1.5:1 to about 1:8. In an aspect the molar ratio of total carboxyl functional groups (—COOH) to the activating agent is from about 1:5:1 to about 1:5. In an aspect, the molar ratio of total carboxyl functional groups (—COOH) to the activating agent is about 1.5:10, about 1.5:9, about 1.5:8, about 1.5:7, about 1.5:6, about 1.5:5, about 1.5:4, about 1.5:3, about 1.5:2, or about 1.5:1.

In an aspect, the molar ratio of total carboxyl functional groups (—COOH) to total hydroxyl functional groups (—OH) is from about 1.5:1 to about 1:1.5. In an aspect, the ratio of total carboxyl functional groups (—COOH) to total hydroxyl functional groups (—OH) is from about 1.25:1 to about 1:1.25. In an aspect, the molar ratio of total carboxyl functional groups (—COOH) to total hydroxyl functional groups (—OH) is about 1.5:1, about 1.45:1.05, about 1.4:1.1, about 1.35:1.15, about 1.3:1.2, about 1.25:1.25, about 1.2:1.3, about 1.1:1.4, about 1.05:1.45, or about 1:1.5.

### 2. Elastomer Compositions

In an aspect, a composition is prepared by combining the elastomer with one or more solvents to form a gel or a paste.

In an aspect, the elastomer is crumbled to form an elastomer powder. In an aspect, the crosslinked polyester elastomer is crumbled to form a crosslinked polyester elastomer powder.

In an aspect, a composition is prepared by shearing the elastomer with a solvent, as described herein, to form a sheared gel. In another aspect, a composition is prepared by first combining the elastomer, as described herein, with a solvent thereby forming a mixture and then shearing the mixture.

In an aspect, the composition is a gel or a paste. In an aspect, the composition is a gel.

Gel compositions according to the present invention are characterized by the oscillation amplitude as well as oscillation frequency-dependent rheology tests at 25° C. In the linear viscoelastic region within the frequency range from 0.01-100 Hz, a gel has a storage modulus G′ which is always greater than the loss modulus G″. G′ and G″ here are rheological parameters known to the person skilled in the art. The elastic or storage modulus, designated as G′, is an indicator of how elastic the material is i.e., how much mechanical energy is being stored per cycle of deformation whereas, the viscous or loss modulus, namely G″, is the measure of the lost or dissipated mechanical energy as heat and/or other form per cycle of deformation and they collectively quantify the elastic or viscous fraction of viscoelastic solids and/or liquids and are described for example in Ferry, J. D., Viscoelastic Properties of Polymers, John Wiley & Sons, Inc. New York, 1980. ISBN 0-471-04894-1

The polyester elastomer gels according to the invention have an excellent yield point which, for example, has an advantageous effect on their thickening properties and also their ability to stabilize dispersed constituents of personal care formulations. For dynamic oscillation rheology tests, for example a DHR hybrid rheometer (TA-Instruments) equipped with a 25 mm parallel plate steel geometry can be used.

Polyester elastomer gels are notable for the fact that, at a shear rate of 10 s⁻¹ and a temperature of 25° C., they have a viscosity of less than 100,000,000 cp and at the same time satisfies G′>G″; Tan-δ>1 within the linear viscoelastic region demonstrating a frequency nearly invariant characteristics. The polyester gels prepared by the methods described herein are characterized by good flowability, which has an advantageous effect on their handleability and processability, but nevertheless have a pronounced yield point and therefore good thickening and stabilizing properties.

In an aspect, the modulus (G′) of the gel is from about 10 Pa to about 100,000 Pa as measured by rheometer within linear viscoelastic region using dynamic rheology. In an aspect, the modulus (G′) of the gel is from about 100 Pa to about 50,000 Pa. In an aspect, the modulus (G′) of the gel is from about 500 Pa to about 30,000 Pa, In an aspect, the modulus (G′) of the gel is about 10 Pa, about 100 Pa, or about 500 Pa, or about 700 Pa, or about 800 Pa, or about 1,000 Pa, or about 1,500 Pa, or about 2,000 Pa, or about 2,500 Pa, or about 5,000 Pa, or about 10,000 Pa, or about 15,000 Pa, or about 25,000 Pa, or about 50,000 Pa, or about 100,000 Pa.

In an aspect, the gel is comprised of particles of size from about 1 μm to about 500 μm as measured by optical microscope. In an aspect, the gel is comprised of particles of size from about 25 μm to about 400 μm. In an aspect, the gel is comprised of particles of size of about 1 μm, about 5 μm, about 10 μm, about 15 μm, about 20 μm, about 25 μm, about 30 μm, about 35 μm, about 40 μm, about 45 μm, about 50 μm, about 75 μm, about 100 μm, about 125 μm, about 150 μm, about 175 μm, about 200 μm, about 225 μm, about 250 μm, about 275 μm, about 300 μm, about 325 μm, about 350 μm, about 375 μm, about 400 μm, or about 500 μm.

In an aspect, the viscosity of the gel is from about 10 cp to about 1,000,000 cp as measured by rheometer at a shear rate of 10 s¹. In an aspect, the viscosity of the gel is from about 30,000 cp to about 500,000 cp. In an aspect, the viscosity of the gel is about 10 cp, about 1,000 cp, about 5,000 cp, about 10,000 cp, about 15,000 cp, about 20,000 cp, about 25,000 cp, about 30,000 cp, about 35,000 cp, about 40,000 cp, about 45,000 cp, about 50,000 cp, about 55,000 cp, about 60,000 cp, about 65,000 cp, about 70,000 cp, about 75,000 cp, about 80,000 cp, about 85,000 cp, about 90,000 cp, about 95,000 cp, about 100,000 cp, about 150,000 cp, about 200,000 cp, about 250,000 cp, about 300,000 cp, about 350,000 cp, about 400,000 cp, about 450,000 cp, about 500,000 cp, about 550,000 cp, about 600,000 cp, about 650,000 cp, about 700,000 cp, about 750,000 cp, about 800,000 cp, about 850,000 cp, about 900,000 cp, about 950,000 cp, or about 1,000,000 cp.

In an aspect, the elastomer composition is prepared using the methods described herein.

### III. Methods

### A. Methods of Preparing an Elastomer

### 1. Esterification Reaction

In an aspect, the present disclosure is directed to a method of preparing an elastomer comprising reacting:
(i) at least one activated polycarboxylic acid; and
(ii) at least one polyol.

In an aspect, the present disclosure is directed to a method of preparing an elastomer comprising:
(a) reacting:
   (i) at least one activated polycarboxylic acid; and
   (ii) at least one polyol; in the absence of solvent in a first reaction stage; and
(b) continuing the reaction in presence of at least one solvent in a second reaction stage.

In an aspect, the present disclosure is directed to a method of preparing an elastomer comprising:
(a) reacting:
   (i) at least one activated polycarboxylic acid; and
   (ii) at least one polyol, in absence of solvent in a first reaction stage; and
(b) continuing the reaction in presence of at least one solvent in second reaction stage;
wherein first reaction stage is for a period of at least 2 hours.

In an aspect, the at least one activated polycarboxylic acid is a compound generated in-situ or ex-situ by reacting a polycarboxylic acid of formula (III) as described herein with an activating agent. In an aspect, the at least one activated polycarboxylic acid is a compound generated in-situ by reacting a polycarboxylic acid of formula (III) as described herein with an activating agent.

In an aspect, at least one polyol is a compound of formula (II) as described herein.

In an aspect, at least one activating agent is a compound of formula (IV) as described herein.

In an aspect, the preparation of the elastomer is under nitrogen protection, with vacuum, and combinations thereof.

In an aspect, the method further comprises the addition of water to quench the activating agent from the reaction.

In an aspect, the method further comprises:
(iii) combining the crosslinked polyester structure with a second solvent thereby forming a swollen crosslinked polymer structure;
(iv) subjecting the swollen crosslinked polymer structure to shear force thereby forming a uniform polyester elastomer.

In an aspect, the shear force is provided by any type of mixing and shearing equipment. In an aspect, the mixing and shearing equipment is batch mixer, planetary mixer, single or multiple screw extruder, dynamic or static mixer, colloid mill, homogenizer, sonolator, or a combination thereof.

### 2. By-Product Removal

In an aspect, the method further comprises removing water and alcohol by-product from the reaction. In a further aspect, the water and alcohol by-products are removed from the reaction by mixing and heating the reaction. In an aspect, the reaction is heated to above about 120° C. In an aspect, the water and alcohol by-products are removed from the reaction by nitrogen flow or by vacuum. 3. Catalyst

In an aspect, the reaction further comprises a catalyst. Such catalysts include, without limit Yb(OTf)₃, Sc(OTf)₃, Hf(OTf)₄, Bi(OTf)₃, Al(OTf)₃, Zn(OTf)₂, Mg(ClO₄)₂, Cu(OTf)₂, Ti(OCH(CH₃)₂)₄, 1,8-diazabicyclo(5.4.0)undec-7-ene (DBU), pyridine, 4-dimethylaminopyridine (DMAP).

### 4. Solvent

In an aspect, the reaction occurs in the presence of a solvent. In some aspects, the solvent is biobased or naturally derived. In an aspect, the solvent is a triglyceride solvent, a mono-ester solvent, a di-ester solvent, a citrate ester solvent, an ether solvent, a carbonate solvent, a hydrocarbon solvent, a silicone solvent, and combinations thereof.

In an aspect, the solvent is a triglyceride solvent of formula (V)

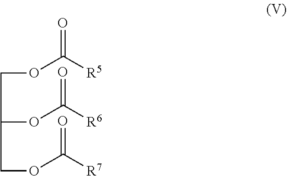

wherein
each R⁵, R⁶, and R⁷ are independently C₁-C₃₅ alkyl group, C₁-C₃₅ heteroalkyl group, C₂-C₃₅ alkene group, or C₂-C₃₅ heteroalkene group.

In an aspect, the solvent is of formula (V), wherein R⁵, R⁶, and R⁷ are independently C₂-C₁₇ alkyl group or C₂-C₁₇ alkylene group.

In an aspect, the solvent is a triglyceride solvent selected from the group consisting of caprylic/capric triglyceride, triheptanoin, corn oil, soybean oil, olive oil, rape seed oil, cotton seed oil, coconut oil, almond oil, argon oil, rosehip oil, black seed oil, grape seed oil, avocado oil, apricot kernel oil, geranium oil, lavender oil, rosehip oil, macadamia oil, eucalyptus oil, sardine oil, herring oil, safflower oil, linseed oil, sunflower oil, olive oil, canola oil, sesame oil, cottonseed oil, palm oil, rapeseed oil, tung oil, fish oil, peanut oil, cuphea oil, milkweed oil, salicornia oil, whale oil, castor oil, and combinations thereof. In an aspect, the triglyceride solvent is selected from the group consisting of caprylic/capric triglyceride, triheptanoin, and combinations thereof.

In an aspect, the solvent is a mono-ester solvent of formula (VI)

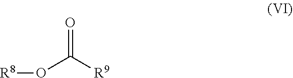

wherein
each R⁸ and R⁹ are independently C₁-C₃₅ alkyl group, C₁-C₃₅ heteroalkyl group, C₂-C₃₅ alkene group, or C₂-C₃₅ heteroalkene group.

In an aspect, the solvent is a mono-ester solvent of formula (VI), wherein R⁸ is C₅-C₁₇ alkyl group or C₅-C₁₇ alkene group and R⁹ is C₂-C₁₇ alkyl group or C₂-C₁₇ alkene group.

In an aspect, the solvent is a mono-ester solvent selected from the group consisting of coco-caprylate, coco-caprate, jojoba oil, jojoba esters, isopropyl jojobate, ethyl macadamiate, isoamyl laurate, heptyl undecylenate, methylheptyl isostearate, isostearyl isostearate, glyceryl ricinoleate, isostearyl palmitate, myristyl myristate, octyldodecyl myristate, octyldodecyl hydroxystearate, butyl myristate, ethylhexyl cocoate, ethylhexyl palmitate, ethylhexyl stearate, butyl stearate, decyl oleate, isocetyl behenate, isocetyl myristate, isocetyl palmitate, isocetyl stearate, isodecyl oleate, isopropyl isostearate, isopropyl myristate, isopropyl palmitate, oleyl oleate, propylene glycol laurate, octyldodecyl erucate, C₁₂-C₁₃ alkyl lactate, C₁₂-C₁₅ alkyl lactate, isostearyl lactate, glycereth-5-lactate, lauryl lactate, myristyl lactate, oleyl lactate, laureth-2-benzoate, C₁₂-C₁₅ alkyl benzoate, C₁₂-C₁₅ pareth-3-benzoate, dipropylene glycol benzoate, isodecyl salicylate, C₁₂-C₁₅ alkyl salicylate, tridecyl salicylate, ethylhexyl isononanoate, cetyl ethylhexanoate, isononyl isononanoate, isodecyl ethylhexanoate, isodecyl isononanoate, tridecyl ethylhexanoate, isotridecyl isononanoate, isostearyl isononanoate, cetearyl isononanoate, laureth-2-ethylhexanoate, cetearyl ethylhexanoate, isodecyl neopentanoate, isostearyl neopentanoate, nyristyl neopentanoate, isostearyl behenate, octyldodecyl neopentanoate, tridecyl neopentanoate, and combinations thereof. In an aspect, the mono-ester solvent is selected from the group consisting of coco-caprylate/caprate, coco-caprylate, jojoba oil, isoamyl laurate, methylheptyl isostearate, C₁₂-C₁₃ alkyl lactate, C₁₂-C₁₅ alkyl lactate, lauryl lactate, ethylhexyl isononanoate, cetyl ethylhexanoate, isononyl isononanoate, isodecyl ethylhexanoate, isodecyl isononanoate, tridecyl ethylhexanoate, isotridecyl isononanoate, isostearyl isononanoate, cetearyl isononanoate, and combinations thereof.

In an aspect, the mono-ester solvent is selected from the group consisting of coco-caprylate/caprate, coco-caprylate, isoamyl laurate, isononyl isononanoate, heptyl undecylenate, jojoba oil, jojoba esters, and combinations thereof.

In an aspect, the solvent is:
(a) a di-ester solvent of formula (VII)

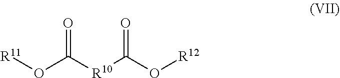

wherein
R¹⁰ is C₁-C₃₅ alkyl group, C₁-C₃₅ heteroalkyl group, C₂-C₃₅ alkene group, or C₂-C₃₅ heteroalkene group; and
R¹¹ and R¹² are independently C₁-C₃₅ alkyl group, C₁-C₃₅ heteroalkyl group, C₂-C₃₅ alkene group, or C₂-C₃₅ heteroalkene group; or
(b) a di-ester solvent of formula (VIII)

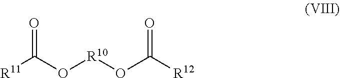

wherein
R¹⁰ is C₁-C₃₅ alkyl group, C₁-C₃₅ heteroalkyl group, C₂-C₃₅ alkene group, or C₂-C₃₅ heteroalkene group; and
R¹¹ and R¹² are independently H, C₁-C₃₅ alkyl group, C₁-C₃₅ heteroalkyl group, C₂-C₃₅ alkene group, or C₂-C₃₅ heteroalkene group; or
(c) a di-ester solvent of formula (IX)

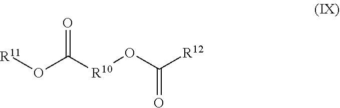

wherein
R¹⁰ is C₁-C₃₅ alkyl group, C₁-C₃₅ heteroalkyl group, C₂-C₃₅ alkene group, or C₂-C₃₅ heteroalkene group; and
R¹¹ and R¹² are independently C₁-C₃₅ alkyl group, C₁-C₃₅ heteroalkyl group, C₂-C₃₅ alkene group, or C₂-C₃₅ heteroalkene group.

In an aspect, the solvent is a di-ester solvent of formula (VII), formula (VIII), or formula (IX), wherein R¹⁰ is C₂-C₁₀ alkyl group or C₂-C₁₀ alkene group and R¹¹ and R¹² are independently C₁-C₁₂ alkyl group or C₂-C₁₂ alkene group.

In an aspect, the di-ester solvent is selected from the group consisting of diethyl succinate, dibutyl succinate, diethyhexyl succinate, diisopropyl sebacate, dimethyl sebacate, diethyl sebacate, dibutyl sebacate, diisostearyl dimer, diisostearyl malate, isostearyl stearoyl stearate, isocetyl stearoyl stearate, octyldodecyl stearoyl stearate, diethylhexyl malate, diethylhexyl maleate, dipropylene glycol dibenzoate, dicapryl adipate, dicaprylyl maleate, diisopropyl dimer, diisopropyl adipate, diisobutyl adipate, diisopropyl sebacate, diisostearyl dimer, diethyhexyl succinate, diethylene glycol diethylhexanoate, neopentyl glycol dicaprate, propylene glycol dicaprylate/dicaprate, neopentyl glycol diisostearate, neopentyl glycol diethylhexanoate, neopentyl glycol diheptanoate, and combinations thereof.

In an aspect, the di-ester solvent is selected from the group consisting of dicapryl adipate, dicaprylyl maleate, diisopropyl adipate, diisobutyl adipate, diethyl succinate, dibutyl succinate, diethyhexyl succinate, diisopropyl sebacate, dimethyl sebacate, diethyl sebacate, dibutyl sebacate, neopentyl glycol diethylhexanoate, neopentyl glycol diheptanoate, and combinations thereof.

In an aspect, the solvent is a citrate ester solvent of formula (X)

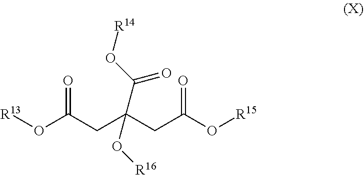

wherein
R¹³, R¹⁴, R, and R¹⁶ are independently H, C₁-C₃₅ alkyl group, C₁-C₃₅ heteroalkyl group, C₂-C₃₅ alkene group, or C₂-C₃₅ heteroalkene group.

In an aspect, the solvent is a citrate ester solvent of formula (X), wherein R¹³, R¹⁴, and R¹⁵ are independently C₁-C₁₀ alkyl group or C₂-C₁₀ alkene group and R¹⁶ is an acetyl group.

In an aspect, the solvent is a citrate ester solvent selected from the group consisting of tricaprylyl citrate, triisostearyl citrate, triisocetyl citrate, trioctyldodecyl citrate, triethyl citrate, tributyl citrate, acetyl triethyl citrate, acetyl tributyl citrate, trioctyldodecyl citrate, triisocetyl citrate, and combinations thereof.

In an aspect, the solvent is an ether solvent of formula (XI)

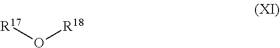

wherein
R¹⁷ and R¹⁸ are independently C₂-C₂₀ alkyl group, C₂-C₂₀ heteroalkyl group, C₂-C₂₀ alkene group, or C₂-C₂₀ heteroalkene group

In an aspect, the solvent is an ether solvent of formula (XI), wherein R¹⁷ and R¹⁸ are independently C₂-C₂₀ alkyl group.

In an aspect, the solvent is an ether solvent selected from the group consisting of dicaprylyl ether, didecyl ether, panthenyl ethyl ether, dicetyl ether, dimyristyl ether, distearyl ether, distearyl ether, dilauryl ether, and combinations thereof.

In an aspect, the ether solvent is selected from the group consisting of dicaprylyl ether, didecyl ether, and combinations thereof.

In an aspect, the solvent is a carbonate solvent of formula (XII)

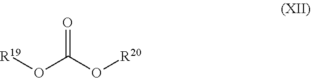

wherein
R¹⁹ and R²⁰ are independently C₂-C₂₀ alkyl group, C₂-C₂₀ heteroalkyl group, C₂-C₂₀ alkene group, or C₂-C₂₀ heteroalkene group.

In an aspect, the solvent is a carbonate solvent of formula (XII), wherein R¹⁹ and R²⁰ are independently C₂-C₂₀ alkyl group.

In an aspect, the solvent is a carbonate solvent selected from the group consisting of dicaprylyl carbonate, diethyl hexyl carbonate, and combinations thereof.

In an aspect, the solvent is a hydrocarbon with number of carbon atoms from C₄ to C₆₀. In another aspect, the solvent is a hydrocarbon with a number of carbon atoms from C₁₀ to C₅₀. In a further aspect, the solvent is a hydrocarbon with a number of carbon atoms from C₂₀ to C₄₀. .

In an aspect, the solvent is a hydrocarbon solvent selected from the group consisting of farnesene, hydrogenated farnesene, coconut alkanes, coconut/palm kernel alkanes, C₉-C₁₂ alkane, C₁₀-C₁₃ alkane, C₁₂-C₁₇ alkane, C₁₃-C₁₄ alkane, C₁₃-C₁₅ alkane, C₁₄-C₁₇ alkane, C₁₄-C₁₉ alkane, C₁₄-C₂₀ alkane, C₁₄-C₂₂ alkane, C₁₅-C₁₉ alkane, C₂₁-C₂₈ alkane, C₁₇-C₂₃ alkane, C₉-C₁₂ isoalkane, C₉-C₁₃ isoalkane, C₉-C₁₄ isoalkane, C₉-C₁₆ isoalkane, C₁₀-C₁₁ isoalkane, C₁₀-C₁₂ isoalkane, C₁₀-C₁₃ isoalkane, C₁₁-C₁₂ isoalkane, C₁₁-C₁₃ isoalkane, C₁₁-C₁₄ isoalkane, C₁₂-C₁₄ isoalkane, C₁₂-C₁₅ isoalkane, C₁₂-C₂₀ isoalkane, C₁₃-C₁₄ isoalkane, C₁₃-C₁₆ isoalkane, C₁₄-C₁₆ isoalkane, C₁₅-C₁₉ isoalkane, C₁₀-C₁₆ olefin, C₁₂-C₁₈ olefin, C₁₈-C₂₆ olefin, C₂₀ olefin, C₂₀-C₂₄ olefin, C₂₄-C₃₀ olefin, C₂₆-C₂₈ olefin, C₂₆-C₅₄ olefin, C₂₈-C₃₆ olefin, C₂₈-C₅₂ olefin, C₃₀-C₃₈ olefin, C₃₀-C₄₅ olefin, C₄-C₁₂ olefin, C₄. C₆ olefin, C₅-C₆ olefin, hydrogenated poly(C₆/C₁₀/C₁₄ olefin), hydrogenated poly(C₆-C₁₂ olefin), hydrogenated poly(C₆-C₁₄ olefin), hydrogenated poly(C₆-C₂₀ olefin), hydrogenated poly(C₈/C₁₂ olefin), poly(C₂₀-C₂₈ olefin), poly(C₃₀-C₄₅ olefin), poly(C₄-C₁₂ olefin), poly(C₆-C₁₄ olefin), hexadecene, C₃₂ alkane, C₃₂ isoalkane, C₅₄ alkane, C₅₄ isoalkane, diethylhexylcyclohexane, undecane, tridecane, tetradecane, pentadecane, hexadecane, octadecane, docosane, squalane, hydrogenated polyisobutene, polybutene, hydrogenated polydecene, hydrogenated didecene, mineral oil, liquidum, petrolatum, dodecane, isohexadecane, isododecane, isoeicosane, and combinations thereof. In an aspect, the hydrocarbon solvent is selected from the group consisting of squalane, farnesene, hydrogenated farnesene, coconut alkanes, C₉-C₁₂ alkane, C₁₃-C₁₅ alkane, C₁₄-C₁₉ alkane, C₁₄-C₂₀ alkane, C₁₄-C₂₂ alkane, C₁₅-C₁₉ alkane, C₁₃-C₁₆ isoalkane, dodecane, undecane, tridecane, tetradecane, pentadecane, hexadecane, hexadecene, octadecane, squalane, isododecane, isohexadecane, C₃₂ alkane, C₃₂ isoalkane, C₅₄ alkane, C₅₄ isoalkane, and combinations thereof. In an aspect, the hydrocarbon solvent is selected from the group consisting of squalane, hydrogenated farnesene, coconut alkanes, C₉-C₁₂ alkane, C₁₃-C₁₅ alkane, C₁₃-C₁₆ isoalkane, C₁₄-C₁₉ alkane, dodecane, tetradecane, isododecane, hexadecane, octadecane, hexadecene, C₃₂ alkane, C₃₂ isoalkane, C₅₄ alkane, C₅₄ isoalkane, and combinations thereof.

In as aspect, the hydrocarbon solvent is selected from the group consisting of squalane, C₃₂ alkane, C₃₂ isoalkane, C₅₄ alkane, C₅₄ isoalkane, and combinations thereof.

In an aspect, the solvent is a silicone solvent selected from the group consisting of dimethicone, phenyl dimethicone, caprylyl methicone, ethyl trisiloxane, cyclotetrasiloxane, cyclopentasiloxane, cyclohexasiloxane, and combinations thereof.

In an aspect, the first solvent is any solvent described herein.

In an aspect, the amount of the first solvent in the reaction is present from about 0% to about 90% of the total weight. In an aspect, the amount of the first solvent present is from about 10% to about 85% of the total weight of the carboxylic acid, the alcohol, and the solvent. In an aspect, the amount of the first solvent present is about 10%, about 15%, about 20%, about 25%, about 30%, about 35%, about 40%, about 45%, about 50%, about 55%, about 60%, about 65%, about 70%, about 75%, about 80%, or about 85% of the total weight.

In an aspect, the second solvent is any solvent described herein.

In an aspect, the amount of the second solvent in the reaction is present from about 0% to about 90% of the total weight. In an aspect, the amount of the second solvent present is from about 10% to about 85% of the total weight of the carboxylic acid, the alcohol, and the solvent. In an aspect, the amount of the second solvent present is about 10%, about 15%, about 20%, about 25%, about 30%, about 35%, about 40%, about 45%, about 50%, about 55%, about 60%, about 65%, about 70%, about 75%, about 80%, or about 85% of the total weight.

### 5. Temperature

In an aspect, the reaction occurs at a temperature from about 25° C. to about 150° C. In an aspect, the reaction occurs at a temperature from about 30° C. to about 125° C. or about 40° C. to about 100° C. In an aspect, the reaction occurs at a temperature of about 25° C., about 30° C., about 40° C., about 60° C., about 65° C., about 70° C., about 75° C., about 80° C., about 85° C., about 90° C., about 95° C., about 100° C., about 105° C., about 110° C., or about 150° C.

### 6. Time

In an aspect, the reaction time is from about 1 hour to about 72 hours. In an aspect, the reaction time is from about 6 hours to about 24 hours. In an aspect, the reaction time is from about 8 hours to about 27 hours. In an aspect, the reaction time is about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, about 12 hours, about 12.5 hours, about 13 hours, about 13.5 hours, about 14 hours, about 14.5 hours, about 15 hours, about 15.5 hours, about 16 hours, about 16.5 hours, about 17 hours, about 17.5 hours, about 18 hours, about 18.5 hours, about 19 hours, about 19.5 hours, about 20 hours, about 20.5 hours, about 21 hours, about 21.5 hours, about 22 hours, about 22.5 hours, about 23 hours, about 23.5 hours, about 24 hours, about 24.5 hours, about 25 hours, about 25.5, hours about 26 hours, about 26.5 hours, or about 27 hours.

### 7. Gel or Paste

In an aspect, the elastomer described herein is used to prepare a gel, paste, film, or powder composition by the methods described herein. In an aspect, the uniform polyester elastomer is crumbled to form an elastomer powder. In an aspect, the uniform polyester elastomer is processed into a gel or a paste. In an aspect, the uniform polyester elastomer is a gel.

In an aspect, a polyester elastomer is mixed with a solvent before being processed to make a gel.

In an aspect, a polyester elastomer is swelled in a solvent before being processed to make a gel. In an aspect, the time of polyester elastomer swelling in a solvent is from 1 hour to 1 week.

In an aspect, polyester elastomer and solvent mixture is processed by homogenizer to produce a gel.

In an aspect, the viscosity, modulus, and particle size of the gel are described herein.

### IV. Personal Care Formulations

In an aspect of the present disclosure, the elastomers compositions described herein is incorporated into a personal care formulation. In an aspect, the gels prepared from the elastomers described herein are incorporated into a personal care formulation.

In an aspect, the personal care formulation further comprises a preservative, an antioxidant, a chelating agent, a gum or thickener, an oil, a wax, a fragrance, an essential oil, an emulsifier, a surfactant, and combinations thereof.

In an aspect, the personal care formulation is a deodorant, an antiperspirant, a skin cream, a facial cream, a hair shampoo, a hair conditioner, a mousse, a hair styling gel, a hair spray, a protective cream, a lipstick, a facial foundations, blushes, makeup, a mascara, a skin care lotion, a moisturizer, a facial treatment, a personal cleanser, a facial cleanser, a bath oil, a perfume, a shaving cream, a pre-shave lotion, an after-shave lotion, a cologne, a sachet, or a sunscreen formulation.

### V. Additional Formulations

In an aspect, the products of the present disclosure, i.e., the crosslinked polyester elastomer, is added to formulations comprising pharmaceuticals, biocides, herbicides, pesticides, or other biologically active substances.

In an aspect, the products of the present disclosure, i.e., the crosslinked polyester elastomer, are used to incorporate water and water-soluble substances into hydrophobic systems. In an aspect, the products of the present disclosure, i.e., the crosslinked polyester elastomer, modify the rheological, physical or energy absorbing properties of oil phases in either their neat or finished condition.

### VI. Use of the Gels

In one aspect, the disclosure relates to the use of a gel described herein for personal care formulations.

### Examples

The following examples are included to demonstrate various aspects of the present disclosure. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the present disclosure, appreciate that many changes can be made in the specific examples which are disclosed and still obtain a like or similar result without departing from the spirit and scope of the disclosure.

### Example 1: Esterification to Prepare Polyester Elastomer

Hydrogenated castor oil (141.0 g, 150.07 mmol), hydrogenated dimer acid (109.1 g, 194.50 mmol) were added into a N₂-flushed reactor equipped with a heating mantle, overhead stirrer, reflux condenser, addition funnel, and thermocouple. The stirred contents were heated to 100° C. for 1 hour. Titanium (IV) isopropoxide (1.38 g, 3.5 mmol) was added and the stirring was continued at 100° C. To this mixture, Di-tert-butyl dicarbonate, (DIBOC) (55.2 g, 252.92 mmol) was added and the stirring was continued for another 4.5 hours. At this point, the contents of the reaction mixture turned into a viscous gum. The analysis of the viscous gum by ¹H NMR technique indicated about 60% conversion of starting monomers without any side reactions. To further facilitate the reaction, an additional amount of DIBOC (28.28 g, 129.57 mmol) was added to the stirring mixture and heating continued at 100° C. for 3 hours, when the entire mixture turned into rubber. The rubber was analyzed by FTIR, where complete monomer conversion was indicated by the disappearance of starting OH frequency at 3000-3500 cm⁻¹. The rheology analysis indicated that the rubber had a modulus (G′) of about 16,819 Pa.

### Example 2: Esterification to Prepare Polyester Elastomer

Hydrogenated castor oil (141.0 g, 150.07 mmol) and hydrogenated dimer acid (109.1 g, 194.50 mmol) were added into a N₂-flushed reactor equipped with a heating mantle, overhead stirrer, reflux condenser, addition funnel, and thermocouple. The stirred contents were heated to 100° C. for 1 hour. Titanium (IV) isopropoxide (1.38 g, 3.5 mmol) was added and the stirring was continued at 100° C. To this mixture, Di-tert-butyl dicarbonate, (DIBOC) (55.2 g, 252.92 mmol) was added and the stirring continued for another 4.5 hours when the entire reaction mixture turned into a viscous gum. At this point, hemisqualane (500.0 g) and DIBOC (22.5 g, 103.09 mmol) were added to the mixture and stirring continued at 75° C. for 5 hours, when the entire mixture turned into an elastomer powder. Deionized (DI) water (5.0 g) was added and stirred for 1 hour at 75° C. The unreacted water and volatile byproducts were stripped off under low pressure at 75° C. Detailed characterization of the powder sample using FTIR method indicated complete conversion of monomers to crosslinked polyester. The rheology analysis of the powder sample indicated that the rubber had a modulus (G′) of about 16,017 Pa.

### Example 3: Esterification to Prepare Polyester Elastomer

Hydrogenated castor oil (141.0 g, 150.08 mmol) and hydrogenated dimer acid (109.1 g, 194.50 mmol) were added into a N₂-flushed reactor equipped with a heating mantle, overhead stirrer, reflux condenser, addition funnel, and thermocouple. The stirred contents were heated to 100° C. for 1 hour. Titanium (IV) isopropoxide (1.38 g, 4.8 mmol) was added and the stirring continued at 100° C. To this mixture, Di-tert-butyl dicarbonate, (DIBOC) (55.2 g, 252.92 mmol) was added and the stirring continued for another 4.5 hours when the entire reaction mixture turned into a viscous gum. At this point, hemisqualane (500.0 g) and DIBOC (22.5 g, 103.09 mmol) were added to the mixture and stirring continued at 75° C. After 5 hours, additional DIBOC (22.5 g 103.09 mmol) was added and mixed for 2.5 hours, when the entire mixture turned into a tacky elastomer powder. Deionized (DI) water (5.0 g) was added and stirred for 1 hour at 75° C. The unreacted water and volatile byproducts were stripped off under low pressure at 75° C. The rheology analysis of the powder sample indicated that the rubber had a modulus (G′) of about 3829.7 Pa.

### Example 4: Esterification to Prepare Polyester Elastomer

Hydrogenated castor oil (141.0 g, 150.08 mmol) and hydrogenated dimer acid (109.1 g, 194.50 mmol) were added into a N₂-flushed reactor equipped with a heating mantle, overhead stirrer, reflux condenser, addition funnel, and thermocouple. The stirred contents were heated to 100° C. for 1 hour. Titanium (IV) isopropoxide (1.38 g, 4.8 mmol) was added and the stirring was continued at 100° C. To this mixture, Di-tert-butyl dicarbonate, (DIBOC) (55.2 g, 252.92 mmol) was added and the stirring continued for another 4.5 hours when the entire reaction mixture turned into a viscous gum. At this point, hemisqualane (500.0 g) and DIBOC (22.5 g, 103.09 mmol) were added to the mixture and stirring continued at 75° C. After 5 hours, additional DIBOC (22.5 g 103.09 mmol) was added and mixed for 4.5 hours, when the entire mixture turned into a soft elastomer powder. Deionized (DI) water (5.0 g) was added and stirred for 1 hour at 75° C. The unreacted water and volatile byproducts were stripped off under low pressure at 75° C. The rheology analysis of the powder sample indicated that the rubber had a modulus (G′) of about 5135.6 Pa.

### Example 5: Esterification to Prepare Polyester Elastomer

Hydrogenated castor oil (141.0 g, 150.08 mmol) and hydrogenated dimer acid (109.1 g, 194.50 mmol) were added into a N₂-flushed reactor equipped with a heating mantle, overhead stirrer, reflux condenser, addition funnel, and thermocouple. The stirred contents were heated to 100° C. for 1 hour. Titanium (IV) isopropoxide (1.38 g, 4.8 mmol) was added and stirring continued at 100° C. To this mixture, Di-tert-butyl dicarbonate, (DIBOC) (55.2 g, 252.92 mmol) was added and stirring continued for another 4.5 hours when the entire reaction mixture turned into a viscous gum. At this point, hemisqualane (500.0 g) and DIBOC (22.5 g, 103.09 mmol) were added to the mixture and stirring continued at 75° C. After 5 hours, additional DIBOC (22.5 g, 103.09 mmol) was added and mixed for 5.5 hours, when the entire mixture turned into a soft elastomer powder. Deionized (DI) water (5.0 g) was added and stirred for 1 hour at 75° C. The unreacted water and volatile byproducts were stripped off under low pressure at 75° C. The rheology analysis of the powder sample indicated that the rubber had a modulus (G′) of about 5900.8 Pa.

### Example 6: Esterification to Prepare Polyester Elastomer

Hydrogenated castor oil (141.0 g, 150.08 mmol) and hydrogenated dimer acid (109.1 g, 194.50 mmol) were added into a N₂-flushed reactor equipped with a heating mantle, overhead stirrer, reflux condenser, addition funnel, and thermocouple. The stirred contents were heated to 100° C. for 1 hour. Titanium (IV) isopropoxide (1.38 g, 4.8 mmol) was added and stirring continued at 100° C. To this mixture, Di-tert-butyl dicarbonate, (DIBOC) (55.2 g, 252.92 mmol) was added and stirring continued for another 4.5 hours when the entire reaction mixture turned into a viscous gum. At this point, hemisqualane (500.0 g) and DIBOC (22.5 g, 103.09 mmol) were added to the mixture and stirring continued at 75° C. After 5 hours, additional DIBOC (22.5 g 103.09 mmol) was added and mixed for 6.5 hours, when the entire mixture turned into a soft elastomer powder. Deionized (DI) water (5.0 g) was added and stirred for 1 hour at 75° C. The unreacted water and volatile byproducts were stripped off under low pressure at 75° C. The rheology analysis of the powder sample indicated that the rubber had a modulus (G′) of about 7749.9 Pa.

### Example 7: Esterification to Prepare Polyester Elastomer

Hydrogenated castor oil (141.0 g, 150.08 mmol) and hydrogenated dimer acid (109.1 g, 194.50 mmol) were added into a N₂-flushed reactor equipped with a heating mantle, overhead stirrer, reflux condenser, addition funnel, and thermocouple. The stirred contents were heated to 100° C. for 1 hour. Titanium (IV) isopropoxide (1.38 g, 4.8 mmol) was added and stirring continued at 100° C. To this mixture, Di-tert-butyl dicarbonate (DIBOC) (55.2 g, 252.92 mmol) was added and stirring continued for another 4.5 hours when the entire reaction mixture turned into a viscous gum. At this point, hemisqualane (500.0 g) and DIBOC (22.5 g, 103.09 mmol) were added to the mixture and stirring continued at 75° C. After 5 hours, additional DIBOC (22.5 g, 103.09 mmol) was added and mixed for 11.5 hours, when the entire mixture turned into a non-tacky elastomer powder. Deionized (DI) water (5.0 g) was added and stirred for 1 hour at 75° C. The unreacted water and volatile byproducts were stripped off under low pressure at 75° C. The rheology analysis of the powder sample indicated that the rubber had a modulus (G′) of about 8657 Pa.

### Example 8: Esterification to Prepare Polyester Elastomer

Hydrogenated castor oil (141.0 g, 150.08 mmol) and hydrogenated dimer acid (109.1 g, 194.50 mmol) were added into a N₂-flushed reactor equipped with a heating mantle, overhead stirrer, reflux condenser, addition funnel, and thermocouple. The stirred contents were heated to 100° C. for 1 hour. Titanium (IV) isopropoxide (1.38 g, 4.8 mmol) was added and stirring continued at 100° C. To this mixture, Di-tert-butyl dicarbonate (DIBOC) (55.2 g, 252.92 mmol) was added and stirring continued for another 4.5 hours when the entire reaction mixture turned into a viscous gum. At this point, hemisqualane (500.0 g) and DIBOC (22.5 g, 103.09 mmol) were added to the mixture and stirring continued at 75° C. for 6 hours. Additional DIBOC (11.25 g, 51.54 mmol) was added and mixed for 1.5 hours, when the entire mixture turned into an elastomer powder. The rheology analysis of the powder sample indicated that the rubber had a modulus (G′) of about 11,106 Pa. Deionized (DI) water (5.0 g) was added and stirred for 1 hour at 75° C. The unreacted water and volatile byproducts were stripped off under low pressure at 75° C. Caprylic/capric triglyceride (GTCC) (1400 g) was added and homogenized at room temperature to obtain a soft spreadable gel. Particle size analysis of the gel using optical microscopic analysis showed that the gel has particles with a diameter ranging from 2 to 50 micron.

### Example 9: Esterification to Prepare Polyester Elastomer

Hydrogenated castor oil (133.86 g, 142.48 mmol) and hydrogenated dimer acid (109.1 g, 194.50 mmol) were added into a N₂-flushed reactor equipped with a heating mantle, overhead stirrer, reflux condenser, addition funnel, and thermocouple. The stirred contents were heated to 100° C. for 1 hour. Titanium (IV) isopropoxide (1.38 g, 4.8 mmol) was added and stirring continued at 100° C. To this mixture, Di-tert-butyl dicarbonate (DIBOC) (55.2 g, 252.92 mmol) was added and stirring continued for another 6 hours when the entire reaction mixture turned into a viscous gum. At this point, HARMONIE Soft Fluid (C₉-C₁₂ alkane) (500.0 g) and DIBOC (33.75 g, 154.63 mmol) was added to the mixture and stirring continued at 75° C. for 5 hours. Additional DIBOC (11.25 g, 51.54 mmol) was added and mixed for 4 hour, when the entire mixture turned into an elastomer powder. Deionized (DI) water (5.0 g) was added and stirred for 1 hour at 75° C. The unreacted water and volatile byproducts were stripped off under low pressure at 75° C. The rheology analysis of the powder sample indicated that the rubber had a modulus (G′) of about 5229 Pa.

### Example 10: Esterification to Prepare Polyester Elastomer

Hydrogenated castor oil (133.86 g, 142.48 mmol) and hydrogenated dimer acid (109.1 g, 194.50 mmol) were added into a N₂-flushed reactor equipped with a heating mantle, overhead stirrer, reflux condenser, addition funnel, and thermocouple. The stirred contents were heated to 100° C. for 1 hour. Titanium (IV) isopropoxide (1.38 g, 4.8 mmol) was added and stirring continued at 100° C. To this mixture, Di-tert-butyl dicarbonate (DIBOC) (55.2 g, 252.92 mmol) was added and the stirring continued for another 6 hours when the entire reaction mixture turned into a viscous gum. Additional DIBOC (11.25 g, mmol) was added and mixed for 5 hours. HARMONIE Soft Fluid (C₉-C₁₂ alkane) (250.0 g) and DIBOC (45 g, 103.09 mmol) were added to the mixture and stirring continued at 75° C. for 7 hours, when the entire mixture turned into an elastomer powder. Deionized (DI) water (5.0 g) was added and stirred for 1 hour at 75° C. The unreacted water and volatile byproducts were stripped off under low pressure at 75° C. The rheology analysis of the powder sample indicated that the rubber had a modulus (G′) of about 6741 Pa.

### Example 11: Esterification to Prepare Polyester Elastomer

Hydrogenated castor oil (141.0 g, 150.08 mmol) and hydrogenated dimer acid (109.1 g, 194.50 mmol) were added into a N₂-flushed reactor equipped with a heating mantle, overhead stirrer, reflux condenser, addition funnel, and thermocouple. The stirred contents were heated to 100° C. for 1 hour. Titanium (IV) isopropoxide (1.38 g, 4.8 mmol) was added and stirring continued at 100° C. To this mixture, Di-tert-butyl dicarbonate (DIBOC) (55.2 g, 252.92 mmol) was added and stirring continued for another 4.5 hours when the entire reaction mixture turned into a viscous gum. At this point, Squalane (125.0 g, 295.63 mmol) and DIBOC (45 g, 103.09 mmol) were added to the mixture and stirring continued at 75° C. for 7 hours, when the entire mixture turned into an elastomer powder. Additional DIBOC (22.5 g, 103.09 mmol) was added and continued mixing at 75° C. After about 6 hours, Cetiol LC (397.5 g) and an additional quantity of DIBOC (22.5 g, 103.09 mmol) was added and mixed for 5.5 hours. Deionized (DI) water (5.0 g) was added and stirred for 1 hour at 75° C. The unreacted water and volatile byproducts were stripped off under low pressure at 75° C. The rheology analysis of the powder sample indicated that the rubber had a modulus (G′) of about 8929 Pa.

### Example 12: Esterification to Prepare Polyester Elastomer

Hydrogenated castor oil (141.0 g, 150.08 mmol) and hydrogenated dimer acid (109.1 g, 194.50 mmol) were added into a N₂-flushed reactor equipped with a heating mantle, overhead stirrer, reflux condenser, addition funnel, and thermocouple. The stirred contents were heated to 100° C. for 1 hour. Titanium (IV) isopropoxide (1.38 g, 4.8 mmol) was added and stirring continued at 100° C. To this mixture, Di-tert-butyl dicarbonate (DIBOC) (55.2 g, 252.92 mmol) was added and stirring continued for another 4.5 hours when the entire reaction mixture turned into a viscous gum. At this point, Cetiol LC (125.0 g) and DIBOC (45 g, 206.19 mmol) were added to the mixture and continued stirring at 75° C. for 3 hours, when the entire mixture turned into an elastomer powder. Additional Cetiol LC (125.5 g) and DIBOC (22.5 g, 103.09 mmol) was added and mixed for 2 hours. Deionized (DI) water (5.0 g) was added and stirred for 1 hour at 75° C. The unreacted water and volatile byproducts were stripped off under low pressure at 75° C. The rheology analysis of the powder sample indicated that the rubber had a modulus (G′) of about 15,249.7 Pa.

### Example 13: Esterification to Prepare Polyester Elastomer

Hydrogenated castor oil (218.0 g, 232.0 mmol) and hydrogenated dimer acid (281.82 g, 502.4 mmol) were added into a N₂-flushed reactor equipped with a heating mantle, overhead stirrer, reflux condenser, addition funnel, and thermocouple. The stirred contents were heated to 100° C. for 1 hour. Titanium (IV) isopropoxide (2.76 g, 9.7 mmol) was added and stirring continued at 100° C. To this mixture, Di-tert-butyl dicarbonate (DIBOC) (55.2 g, 252.92 mmol) was added and stirring continued for another 6 hours when the entire reaction mixture turned into a viscous gum. At this point, Cetiol LC (250.0 g) and DIBOC (45 g, 103.09 mmol) were added to the mixture and stirring continued at 75° C. for 3 hours, when the entire mixture turned into an elastomer powder. The rheology analysis of the powder sample indicated that the rubber had a modulus (G′) of about 6945.96 Pa.

### Example 14: Esterification to Prepare Polyester Elastomer

Hydrogenated castor oil (141.0 g, 150.08 mmol) was added into a N₂-flushed reactor equipped with a heating mantle, overhead stirrer, reflux condenser, addition funnel, and thermocouple. The stirred content was heated to 100° C. for 1 hour. Titanium (IV) isopropoxide (1.38 g, 3.5 mmol) was added and stirring continued at 100° C. for 5 minutes. To this mixture, a premix of hydrogenated dimer acid (109.1 g, 194.50 mmol) and Di-tert-butyl dicarbonate (DIBOC) (55.2 g, 252.92 mmol) was added and the stirring continued for another 6 hours when the entire reaction mixture turned into a viscous gum. At this point, Cetiol LC (125.0 g) and DIBOC (45 g, 103.09 mmol) were added to the mixture and stirring continued at 75° C. for 7 hours, when the entire mixture turned into elastomer powder. Deionized (DI) water (5.0 g) was added and stirred for 1 hour at 75° C. The unreacted water and volatile byproducts were stripped off under low pressure at 75° C. The rheology analysis of the powder sample indicated that the rubber had a modulus (G′) of about 26611 Pa.

### Example 15: Swelling of Polyester Elastomer to Prepare Polyester Gel

25 g of the elastomer powder obtained in synthetic Example 11 and Cetiol LC (75 g) were added into a beaker and stirred at room temperature for 2 hours. The mixture was then subjected to high shear mixing using Silverson's high shear rotor/stator laboratory mixer to produce a creamy, translucent gel of very smooth consistency. The optical microscopic analysis of the gel indicated that it has an average particle size of 20 micron. The rheology analysis of the gel sample indicated a modulus (G′) of about 2540 Pa.

### Example 16: Esterification to Prepare Polyester Elastomer

In a suitable vessel equipped with agitation, heat, and an ability to distill off volatile alcohol, 100 g dimer acid was added along with 17 g diglycerol. Next 60 g di-tert-butyl dicarbonate was added as a coupling agent. After all ingredients had been charged under agitation, the temperature was raised to 110° C., and water, t-butanol, and carbon dioxide were stripped off as formed. The temperature was held 12 hours before an additional 60 g di-tert-butyl dicarbonate was added to the vessal. The temperature was held for another 12 hours or until gelation took place and polymer elastomer was formed.

### Example 17: Esterification to Prepare Polyester Elastomer

In a suitable vessel equipped with agitation, heat, and an ability to distill off volatile alcohol, 100 g dimer acid was added along with 17 g diglycerol and 30 g squalane. Next 70 g di-tert-butyl dicarbonate was added as a coupling agent. After all ingredients had been charged under agitation, the temperature was raised to 110° C., and water, t-butanol, and carbon dioxide were stripped off as formed. The temperature was held 12 hours before an additional 70 g di-tert-butyl dicarbonate was added to the vessel. The temperature was held for another 12 hours or until gelation took place and polymer elastomer was formed.

### Example 18: Esterification to Prepare Polyester Elastomer

In a suitable vessel equipped with agitation, heat, and an ability to distill off volatile alcohol, 100 g dimer acid was added along with 17 g diglycerol, 7 g oleic acid, and 30 g squalane. Next 70 g di-tert-butyl dicarbonate (DIBOC) was added as a coupling agent. After all ingredients had been charged under agitation, the temperature was raised to 110° C., and water, t-butanol, and carbon dioxide were stripped off as formed. The temperature was held 12 hours before an additional 70 g di-tert-butyl dicarbonate was added to the vessel. The temperature was held for another 12 hours or until gelation took place and polymer elastomer was formed.

### Comparative Example 19: Esterification to Prepare Polyester Elastomer

Hydrogenated castor oil (141.0 g, 150.07 mmol) and hydrogenated dimer acid (109.1 g, 194.50 mmol) were added into a N₂-flushed reactor equipped with a heating mantle, overhead stirrer, reflux condenser, addition funnel, and thermocouple. The stirred contents were heated to 150° C. for 1 hour. Concentrated sulfuric acid (2.5 g, 2.54 mmol) was added, and stirring continued for 4.5 hours at 100° C. At this point, the color of the reaction mixture changed from light yellow to dark brown. The analysis of the crude reaction mixture by ¹H NMR technique indicated the presence of unwanted side products. To this colored mixture, Cetiol LC (125.0 g) was added and stirring continued at 150° C. for 20 hours. No formation of elastomeric powder was observed at this point. Then the volatile byproducts were stripped off under low pressure at 75° C. The rheology analysis of the crude product indicated that the material had a modulus (G′) of about 7.0 Pa.

### Example 20: Preparation of a Face Serum

Weighed Phase A in the main vessel and mixed under regular stirring until homogeneous. Weighed Phase B and mixed in a vessel until uniform. Added Phase B to the main kettle under homogenization and mixed until uniform. Added Phase C to Phase AB and mixed until uniform. See formulation specifics in Table 1.

TABLE 1

| Phase | Ingredients | INCI Name | % W/W |
|---|---|---|---|
| A | DI water | Water | 63.5 |
| | Xanthan gum | Xanthan gum | 0.5 |
| | Glycerin | Glycerin | 3 |
| | DMDM hydantoin | DMDM hydantoin | 0.5 |
| B | HARMONIE Soft Fluid | C₉-C₁₂ alkane | 5 |
| | Synthetic Example 15 | | 15 |
| | Vitamin E acetate | Tocopherol | 0.5 |
| | DUB ININ | Isononylisononoate | 5 |
| | Sweet almond Oil | Prnus Amygdalus Dulcis | 2 |
| | | (Sweet Almond) Oil | |
| | HARMONIE Luxe | Silica | 4 |
| | Powder | | |
| C | Sepigel 305 | Polyacrylamide & C₁₃-C₁₄ | 1 |
| | | Isoparaffin & Laureth-7 | |

### Example 21: Preparation of a Lotion

Weighed Phase A in the main vessel and mixed under regular stirring until homogeneous and heat up to 70° C. Weighed Phase B and mixed in a vessel until uniform and heat the mixture up to 70° C. Added Phase B to the main kettle under homogenization and mixed until uniform. Added Phase C to Phase AB and mixed until uniform. See formulation specifics in Table 2.

TABLE 2

| Phase | Ingredients | INCI Name | % W/W |
|---|---|---|---|
| A | Deionised water | Water | 61.2 |
| | Cosmedia SP | Sodiumpolyacrylate | 0.3 |
| | Glycerin | Glycerin | 5 |
| | Butylene Glycol | | 2 |
| | DMDM hydantoin | DMDM hydantoin | 0.5 |
| B | Hemisqualane | C₁₃-C₁₅ Alkane | 5 |
| | Synthetic Example 15 | | 15 |
| | Vitamin E acetate | Tocopherol | 0.5 |
| | Cetiol LC | Coco-Caprylate/Caprate | 5 |
| | Cetyl Alcohol | Cetyl Alcohol | 2 |
| | Emulgin SG | Sodium Stearoyl Glutamate | 0.5 |
| C | HARMONIE Lux | Silica | 3 |
| | Powder | | |

### Example 22: Preparation of a Gel Cream

Weighed Phase A in the main vessel and mixed under regular stirring until homogeneous. Weighed Phase B and mixed in a vessel until uniform. Added Phase B to the main kettle under homogenization and mixed until uniform. Added Phase C to Phase AB and mixed until uniform. See formulation specifics in Table 3.

TABLE 3

| Phase | Ingredients | INCI Name | % W/W |
|---|---|---|---|
| A | Deionised water | Water | 79.5 |
| | Sepinov EMT | Sodium polyacrylate | 1.3 |
| | Propylene Glycol | Propylene Glycol | 5 |
| | Butylene Glycol | Butylene glycol | 2 |
| | Sodium Ascorbyl phospate | Sodium Ascorbyl phospate | 3 |
| B | Hemisqualane | C₁₃-C₁₅ Alkane | 3 |
| | Synthetic Example 15 | | 5 |
| | Vitamin E acetate | Tocopherol | 0.5 |
| C | DMDM hydantoin | DMDM hydantoin | 0.7 |

### Example 23: Sheet Mask Base Formula

Made a dispersion of Xanthan gum and carbomer in water, then added the rest of the ingredients of Phase A and q.s. (as much as is sufficient) to 100% with water and homogenized for a short time to make sure all ingredients were well dispersed. Mixed Phase B together until homogeneous, then added into Phase A. Then added Phase C and q.s. to 100% and mixed until homogeneous. See formulation specifics in Table 4.

TABLE 4

| Phase | Ingredients | INCI Name | % W/W |
|---|---|---|---|
| A | KELTROL CG-SFT | Xanthan gum | 0.3 |
| | Glycerin | Glycerin | 3 |
| | Carbopol 941 | Carbomer | 0.3 |
| | Water | Water | q.s. to 100 |
| | DMDM hydantoin | DMDM hydantoin | 0.5 |
| B | Synthetic Example 15 | | 10 |
| | HARMONIE Soft Fluid | C₉-C₁₂ alkane | 5 |
| | Tween 20 | Polysorbate 20 | 0.3 |
| C | Triethanolamine | Triethanolamine | q.s. to 100 |

### Example 24: Preparation of a Color-Correcting (CC) Cream

Heated Phase A to 85 C until all ingredients were dissolved well. Mixed the ingredients of Phase B with a homogenizer. Added Phase C into Phase B and mixed it well. Added Phase BC to Phase A under homogenization and mixed well until uniform. Mixed the ingredients of Phase D and added to Phase ABC dropwise while stirring, ensuring that water phase was well dispersed (q.s. to 10000 with water). Added Phase E while stirring until homogeneous. See formulation specifics in Table 5.

TABLE 5

| Phase | Ingredients | INCI Name | % W/W |
|---|---|---|---|
| A | HARMONIE Mesh | Cyclopentasiloxane (and) PEG/ | 3 |
| | Emulsifier | PPG-20/15 Dimethicone | |
| | Easynov | Octyldodecanol (and) | 3 |
| | | Octydodecyl Xyloside (and) | |
| | | PEG-30 Dipolyhydroxystearate | |
| | Octocrylene | Octocrylene | 2 |
| | Ethylhexyl | Ethylhexyl Methoxycinnamate | 5 |
| | Methoxycinnamate | | |
| | Uvinul A Plus | Diethylamino Hydroxybenzoyl | 2 |
| | | Hexyl Benzoate | |
| | Performalene 400 | Polyethylene | 2 |
| | Caprylic/Capric | Caprylic/Capric Triglyceride | 4 |
| | Triglyceride | | |
| B | Silform 74 | IDD (and) | 5 |
| | | Polymethylsilsesquioxane | |
| | HARMONIE Soft | C₉-C₁₂ Alkane | 8 |
| | Fluid | | |
| | Synthetic Example 15 | | 5 |
| C | Titanium dioxide | Titanium dioxide | 8 |
| | Zin oxide | Zin oxide | 4 |
| | Iron oxide Yellow | Iron oxide Yellow | 0.14 |
| | Iron oxide Red | Iron oxide Red | 0.1 |
| | Iron oxide Black | Iron oxide Black | 0.07 |
| | HARMONIE Lux | Silica | 3 |
| | Powder | | |
| D | DI-water | Water | q.s. |
| | | | to 100 |
| | Disodium EDTA | Disodium EDTA | 0.05 |
| | Sodium Chloride | Sodium Chloride | 1 |
| | Butylene Glycol | Butylene Glycol | 3 |
| E | Euxyl 9010 | Phenoxyethanol (and) | 0.75 |
| | | Ethylhexylglycerin | |

### Example 25: Preparation of a Sunscreen Spray

Mixed Phase A while stirring until homogeneous. Made a premix of Phase B, added to Phase A, and mixed well. Added the ingredients in Phase C to AB one at a time while stirring until homogeneous. See formulation specifics in Table 6.

TABLE 6

| | | | % |
|---|---|---|---|
| Phase | Ingredients | INCI Name | W/W |
| A | Ethylhexyl | Ethylhexyl | 6 |
| | Methoxycinnamate | Methoxycinnamate | |
| | Ethylhexyl Salicylate | Ethylhexyl Salicylate | 5 |
| | Homosalate | Homosalate | 10 |
| | Octocrylene | Octocrylene | 10 |
| | Butyl | Butyl | 5 |
| | Methoxydibenzoylmethane | Methoxydibenzoylmethane | |
| | Tinosorb S | Bis-Ethylhexyloxy Phenol | 6 |
| | | Methoxyphenyl Triazine | |
| | Silsoft 034 | Caprylyl Methicone | 7 |
| | SS 4230 | Cyclopentasiloxane (and) | 2 |
| | | Trimethylsiloxysilicate | |
| | Purolan IDD | Isododecane | 10 |
| | Salacos 99 | Isononyl Isononanoate | 6 |
| | Dub DIPA | Diisopropyl Adipate | 5.7 |
| B | Synthetic Example 15 | | 8 |
| | Cetiol LC | Coco-Caprylate/Caprate | 8 |
| C | DL-Alpha Tocopheryl Acetate | Tocopheryl Acetate | 0.8 |
| | Argan Oil | Argania Spinosa Kernel Oil | 0.5 |
| | Alcohol | Alcohol | 10 |
| Total | | | 100 |

### Example 26: Preparation of a Skin Cream

Mixed Phase A and q.s. to 100% with water while stirring until homogeneous and heated up to 70-75° C. Made a premix of Phase B and heated up to 70-75° C. and mixed well. Made a premix of Phase C and added to Phase B and heated Phase BC until homogenous. Added Phase BC to Phase A under homogenization and mixed well for 5 min. Added Phase D and q.s. to 100%, one at a time to Phase ABC and mixed well. See formulation specifics in Table 7.

TABLE 7

| Phase | Ingredients | INCI Name | % W/W |
|---|---|---|---|
| A | Water | Aqua | q.s. |
| | | | to 100.0 |
| | Keltrol CG-SFT | Xanthan Gum | 0.2 |
| | Glycerin | Glycerin | 0.5 |
| B | Disodium EDTA | Disodium EDTA | 0.05 |
| | Glyceryl Stearate Citrate | Glyceryl Stearate Citrate | 3 |
| | Emulgin SG | Sodium Stearoyl Glutamate | 0.5 |
| | Cutina GMS | Glyceryl Stearate | 1.5 |
| | Lanette O | Cetearyl Alcohol | 2 |
| | Hemisqualane | C₁₃-C₁₅ Alkane | 5 |
| | Cetiol E | PPG-15 Stearyl Ether | 5 |
| C | Synthetic Example 15 | | 5 |
| | Myritol 312 | Caprylic-Capric Triglyceride | 5 |
| D | Preservative | Preservative | q.s. |
| | | | to 100 |
| | Fragrance | Fragrance | q.s. |
| | | | to 100 |

### Example 27: Preparation of a Cream

Mixed Phase A ingredients and q.s. with DM water to 100%, and allowed it to heat to 75 to 80° C. Mixed Phase B ingredients and allowed it to heat to 75 to 80° C. Once the temperature of the both phases reached 75-80° C., homogenized Phase A for 2 min. Slowly added Phase B to Phase A and homogenized for 5 min. Stopped homogenization and started cooling the base under normal stirring. Added phase C ingredients one at a time once the temperature was below 60° C. and mixed well. Added Phase E ingredients one at a time and added to the above mix. Added fragrance to q.s. to 100% below 40° C. and mixed well until homogeneous and stored in an airtight container. See formulation specifics in Table 8.

TABLE 8

| | | | |
|---|---|---|---|
| S. | | | |
| No | Ingredients | INCI Name | % W/W |

Phase A

| | | | |
|---|---|---|---|
| 1 | DM Water | Aqua | q.s. |
| | | | to 100 |
| 2 | EDTA | Disodium EDTA | 0.05 |
| 3 | Niacinamide | Niacinamide | 1 |
| 4 | KOH (20%) | Potassium Hydroxide | 1.5 |

Phase B

| | | | |
|---|---|---|---|
| 5 | DM Water | Aqua | 5 |
| 6 | Butylene Glycol | Butylene Glycol | 1 |
| 7 | Glycerin | Glycerin | 1 |
| 8 | Titanium Dioxide | Titanium Dioxide | 2 |

Phase C

| | | | |
|---|---|---|---|
| 9 | Hystric Acid | Stearic Acid | 16 |
| 11 | BHT | Butylated Hydroxytoluene | 0.02 |
| 12 | Galaxy 610 | Glycol Diistearate | 1.5 |
| 13 | Cetostearyl alcohol | Cetostearyl alcohol | 0.5 |
| 14 | Crodamol IPM | Isopropyl myristate | 3 |

Phase D

| | | | |
|---|---|---|---|
| 15 | Synthetic Example 15 | | 5 |
| 16 | Cetiol Ultimate | Undecane (and) Tridecane | 3 |

Phase E

| | | | |
|---|---|---|---|
| 17 | Novomer EC-2 | Sodium Acrylates/Beheneth-25 | 0.4 |
| | Polymer | Methacrylate Crosspolymer (and) | |
| | | Hydrogenated Polydecene (and) | |
| | | Lauryl Glucoside | |
| 18 | Euxyl 9010 | Phenoxyethanol (and) | 0.5 |
| | | Ethylhexylglycerin | |
| 19 | Fragrance | Fragrance | q.s. |
| | | | to 100 |

### Example 28: Preparation of a Liquid Formulation

Mixed all ingredients in Phase A and q.s. with DM water to 100% until homogeneous and then heated phase A to 75-80° C. Pre-blended phase B ingredients and then added Phase C ingredients to Phase B. Mixed all ingredients in Phase D and heated it up to 75-80° C. Then added Phase BC to Phase D and mixed well and heated the mixture up to 75° C. Once the temperature was attained added Phase BCD to Phase A and mixed under homogenization for 5 minutes. Added Phase E ingredients one at a time into Phase ABCD at a temperature below 60° C. Added fragrance, mixed until homogeneous, and stored in an airtight container. See formulation specifics in Table 9.

TABLE 9

| | | | |
|---|---|---|---|
| S. No | Ingredients | INCI Name | % W/W |

Phase A

| | | | |
|---|---|---|---|
| 1 | DM Water | Aqua | q.s. to 100 |
| 2 | Butylene Glycol | Butylene Glycol | 5 |
| 3 | Xanthan Gum | Xanthan Gum | 0.3 |
| 4 | Silform Hydroflex | Polyacrylate-56 | 8 |
| 5 | Brij ™ S721 | Steareth-21 | 0.7 |

Phase B

| | | | |
|---|---|---|---|
| 6 | Synthetic Example | | 5 |
| | 15 | | |
| 7 | Hemisqualane | C₁₃-C₁₅ Alkane | 3 |

Phase C

| | | | |
|---|---|---|---|
| 8 | Titanium dioxide | Titanium dioxide | 5 |
| 9 | KOBO-BYO-11S2 | CI 77492 (and) | 0.5 |
| | | Triethoxycaprylylsilane | |
| 10 | KOBO-BRO-11S2 | CI 77491 (and) | 0.2 |
| | | Triethoxycaprylylsilane | |
| 11 | KOBO-BBO-11S2 | CI 77499 (and) | 0.001 |
| | | Triethoxycaprylylsilane | |

Phase D

| | | | |
|---|---|---|---|
| 12 | Crodamol GTCC | Caprylic/Capric triglycerides | 3 |
| 13 | Brij ™ S2 | Steareth-2 | 0.35 |
| 14 | Cutina GMS V | Glyceryl Monostearate SE | 0.5 |
| 15 | Ginol 16 (95%) | Cetyl Alcohol | 0.5 |

Phase E

| | | | |
|---|---|---|---|
| 16 | Sepigel 305 | Polyacrylamide & C₁₃-C₁₄ | 0.5 |
| | | Isoparaffin & Laureth-7 | |
| 17 | Euxyl 9010 | Phenoxyethanol (and) | 0.5 |
| | | Ethylhexylglycerin | |
| 18 | Fragrance | Fragrance | 0.25 |

### Example 29: Preparation of a Foundation Cream

Wetted powders with Isononyl Isononanoate. Then added remaining components in Phase A and q.s. with Phase B to 100% and mixed. See formulation specifics in Table 10.

TABLE 10

| Phase | Ingredients | INCI name | % W/W |
|---|---|---|---|
| A | Unipure white LC 987 | CI 77891 (and) | 4.19 |
| | AS-EM | Triethoxycaprylylsilane | |
| | Unipure Yellow 182 | CI 77492 (and) | 0.56 |
| | AS-EM | Triethoxycaprylylsilane | |
| | Unipure Red 381 AS- | CI 77491 (and) | 0.17 |
| | EM | Triethoxycaprylylsilane | |
| | Unipure Black 989 AS- | CI 77499 (and) | 0.08 |
| | EM | Triethoxycaprylylsilane | |
| | Softouch* Boron | Boron Nitride | 2 |
| | Nitride Powder CC6004 | | |
| | HARMONIE Lux | Silica | 3 |
| | Powder | | |
| | DUB ININ | Isononyl Isononanoate | 10 |
| | Cetiol LC | Coco-Caprylate/Caprate | 10 |
| | Synthetic Example 15 | | 70 |
| B | Preservative | Preservative | q.s. to 100 |
| | Fragrance | Fragrance | q.s. to 100 |

### Example 30: Preparation of a Concealer

Mixed all ingredients and heated to 80-90° C. using a Cowles mixer. Ensured the oil thickener is dissolved, then poured the mixture into the jar and let it cool. See formulation specifics in Table 11.

TABLE 11

| Phase | Ingredients | INCI Name | % W/W |
|---|---|---|---|
| A | Synthetic Example 15 | | 70 |
| | Oilkemia 5S polymer | Caprylic/Capric Triglyceride | 5 |
| | | (and) Polyurethane-79 | |
| | Lexfeel Natural | Heptyl Undecylenate | 25 |

### Example 31: Preparation of a Lipstick

Mixed Phase A and q.s. to 100% with castor oil in the main kettle while stirring and heated to 90-95° C. and mixed until uniform. Poured at 75-80° C. into molds. See formulation specifics in Table 12.

TABLE 12

| Phase | Ingredients | INCI Name | % W/W |
|---|---|---|---|
| A | Candelilla wax | Candelilla wax | 13 |
| | Carnauba wax | Carnauba wax | 3 |
| | Beeswax | Beeswax | 2 |
| | Almond oil | Almond oil | 18 |
| | Polyhydroxystearic acid | Polyhydroxystearic acid | 3 |
| | Eutanol G | Octyldodecanol | 5 |
| | Shea butter | Shea butter | 1.5 |
| | Synthetic Example 15 | | 15 |
| | Dye | Dye | 10 |
| | Pigment | Pigment | 5 |
| | Castor oil | | q.s. |
| | | | to 100 |
| | HARMONIE Lux Powder | Silica | 2 |
| | Tocopherol | Tocopherol | 1 |

### OTHER ASPECTS

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific aspects thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and can be applied to the essential features hereinbefore set forth, and follows in the scope of the claimed.

1. An elastomer comprising a reaction product of:
(i) at least one activated polycarboxylic acid; and
(ii) at least one polyol;
wherein (a) at least one activated polycarboxylic acid and at least one polyol have a total of at least five carboxyl and hydroxyl functional groups; and (b) there are at least three carboxyl or hydroxyl functional groups on at least one activated polycarboxylic acid or at least one polyol.

2. The elastomer of claim 1, wherein the at least one activated polycarboxylic acid is a compound of formula (I) 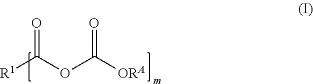
wherein
R¹ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group;
R^{A} is a C₁-C₆₀ monovalent hydrocarbon group; and
m is an integer from 2 to 10.

3. The elastomer of claim 1, wherein the activated polycarboxylic acid is prepared in-situ or ex-situ by reacting a polycarboxylic acid of formula (III) 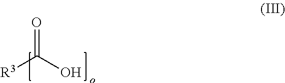
wherein
R³ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
o is an integer from 2 to 10;
with an activating agent.

4. (canceled)

5. The elastomer of claim 3, wherein the polycarboxylic acid of formula (III) is selected from the group consisting of succinic acid, adipic acid, azelaic acid, sebacic acid, dodecanedioic acid, hexadecanedioic acid, C₂₁ dimer acid, C₃₆ dimer acid, hydrogenated C₃₆ dimer acid, aspartic acid, glutamic acid, tartaric acid, malic acid, and combinations thereof.

6. The elastomer of claim 3, wherein the activating agent is a compound of formula (IV) 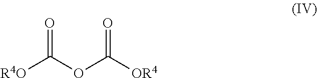
wherein
R⁴ represents a straight-chain or branched alkyl radical with C₁-C₆₀ atoms.

7. The elastomer of claim 6, wherein the activating agent is selected from the group consisting of dimethyl dicarbonate, diethyl dicarbonate, dipropyl dicarbonate, di-tertiary-butyl dicarbonate, and combinations thereof.

8. The elastomer of claim 1, wherein the at least one polyol is a compound of formula (II) 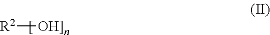
wherein
R² is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
n is an integer from 2 to 10.

9. The elastomer of claim 1, wherein the polyol is selected from the group consisting of glycerol, diglycerol, polyglycerol, sorbitol, castor oil, hydrogenated castor oil, sugar alcohol, monosaccharide, disaccharides, oligosaccharide, polysaccharides, tannin, gallic acid, gluconic acid, lactobionic acid, gluconolactone ethyleneglycol, 1,2-propanediol, 1,3-propanediol, 1,4-butanediol, 1,2-pentanediol, 1,3-pentanediol, 1,4-pentanediol, 1,5-pentanediol, 1,2-hexanediol, 1,5-hexanediol, 1,6-hexanediol, C₃₆ dimer diol, hydrogenated C₃₆ dimer diol, and combinations thereof.

10. The elastomer of claim 6, wherein the molar ratio of total carboxyl functional groups (—COOH) to the activating agent is from about 1.5:1 to about 1:10.

11. The elastomer of claim 1, wherein the molar ratio of total carboxyl functional groups (—COOH) to total hydroxyl functional groups (—OH) is from about 1.5:1 to about 1:1.5.

12. The elastomer of claim 6, wherein the elastomer is prepared by reacting:
(i) at least one polycarboxylic acid of formula (III) 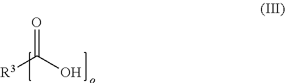
wherein
R³ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
o is an integer from 2 to 10;
(ii) at least one activating agent of formula (IV) 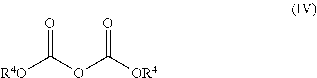
wherein
R⁴ represents a straight-chain or branched alkyl radical with C₁-C₆₀ atoms; and
(iii) at least one polyol of formula (II) 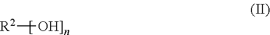
wherein
R² is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
n is an integer from 2 to 10.

13. A method of preparing an elastomer comprising reacting:
(i) at least one activated polycarboxylic acid; and
(ii) at least one polyol.

14. The method of claim 13, wherein the reacting of (i) and (ii) occurs in the presence of a solvent is-selected from the group consisting of a biobased or naturally derived solvent, a triglyceride solvent, a mono-ester solvent, a di-ester solvent, a citrate ester solvent, an ether solvent, a carbonate solvent, a hydrocarbon solvent, a silicone solvent, and combinations thereof.

15. The method of claim 14, wherein the solvent is:
(a) a triglyceride solvent of formula (V) 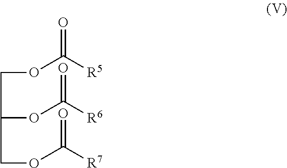
wherein
each R⁵, R⁶, and R⁷ are independently C₁-C₃₅ alkyl group, C₁-C₃₅ heteroalkyl group, C₂-C₃₅ alkene group, or C₂-C₃₅ heteroalkene group; or
(b) a mono-ester solvent of formula (VI) 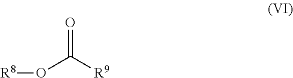
wherein
each R⁸ and R⁹ are independently C₁-C₃₅ alkyl group, C₁-C₃₅ heteroalkyl group, C₂-C₃₅ alkene group, or C₂-C₃₅ heteroalkene group.

16. The method of claim 14, wherein the solvent is:
(a) a triglyceride solvent selected from the group consisting of caprylic/capric triglyceride, triheptanoin, corn oil, soybean oil, olive oil, rape seed oil, cotton seed oil, coconut oil, almond oil, argon oil, rosehip oil, black seed oil, grape seed oil, avocado oil, apricot kernel oil, geranium oil, lavender oil, rosehip oil, macadamia oil, eucalyptus oil, sardine oil, herring oil, safflower oil, linseed oil, sunflower oil, olive oil, canola oil, sesame oil, cottonseed oil, palm oil, rapeseed oil, tung oil, fish oil, peanut oil, cuphea oil, milkweed oil, salicornia oil, whale oil, castor oil, and combinations thereof, or
(b) a mono-ester solvent selected from the group consisting of coco-caprylate, coco-caprate, jojoba oil, jojoba esters, isopropyl jojobate, ethyl macadamiate, isoamyl laurate, heptyl undecylenate, methylheptyl isostearate, isostearyl isostearate, glyceryl ricinoleate, isostearyl palmitate, myristyl myristate, octyldodecyl myristate, octyldodecyl hydroxystearate, butyl myristate, ethylhexyl cocoate, ethylhexyl palmitate, ethylhexyl stearate, butyl stearate, decyl oleate, isocetyl behenate, isocetyl myristate, isocetyl palmitate, isocetyl stearate, isodecyl oleate, isopropyl isostearate, isopropyl myristate, isopropyl palmitate, oleyl oleate, propylene glycol laurate, octyldodecyl erucate, C₁₂-C₁₃ alkyl lactate, C₁₂-C₁₅ alkyl lactate, isostearyl lactate, glycereth-5-lactate, lauryl lactate, myristyl lactate, oleyl lactate, laureth-2-benzoate, C₁₂-C₁₅ alkyl benzoate, C₁₂-C₁₅ pareth-3-benzoate, dipropylene glycol benzoate, isodecyl salicylate, C₁₂-C₁₅ alkyl salicylate, tridecyl salicylate, ethylhexyl isononanoate, cetyl ethylhexanoate, isononyl isononanoate, isodecyl ethylhexanoate, isodecyl isononanoate, tridecyl ethylhexanoate, isotridecyl isononanoate, isostearyl isononanoate, cetearyl isononanoate, laureth-2-ethylhexanoate, cetearyl ethylhexanoate, isodecyl neopentanoate, isostearyl neopentanoate, nyristyl neopentanoate, isostearyl behenate, octyldodecyl neopentanoate, tridecyl neopentanoate, and combinations thereof.

17. The method of claim 14, wherein the solvent is:
(a) a di-ester solvent of formula (VII) 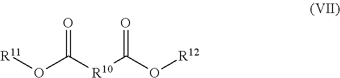
wherein
R¹⁰ is C₁-C₃₅ alkyl group, C₁-C₃₅ heteroalkyl group, C₂-C₃₅ alkene group, or C₂-C₃₅ heteroalkene group; and
R¹¹ and R¹² are independently C₁-C₃₅ alkyl group, C₁-C₃₅ heteroalkyl group, C₂-C₃₅ alkene group, or C₂-C₃₅ heteroalkene group; or
(b) a di-ester solvent of formula (VIII) 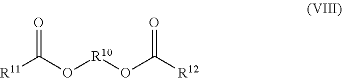
wherein
R¹⁰ is C₁-C₃₅ alkyl group, C₁-C₃₅ heteroalkyl group, C₂-C₃₅ alkene group, or C₂-C₃₅ heteroalkene group; and
R¹¹ and R¹² are independently H, C₁-C₃₅ alkyl group, C₁-C₃₅ heteroalkyl group, C₂-C₃₅ alkene group, or C₂-C₃₅ heteroalkene group; or
(c) a di-ester solvent of formula (IX) 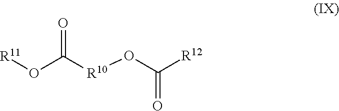
wherein
R¹⁰ is C₁-C₃₅ alkyl group, C₁-C₃₅ heteroalkyl group, C₂-C₃₅ alkene group, or C₂-C₃₅ heteroalkene group; and
R¹¹ and R¹² are independently C₁-C₃₅ alkyl group, C₁-C₃₅ heteroalkyl group, C₂-C₃₅ alkene group, or C₂-C₃₅ heteroalkene group.

18. The method of claim 14, wherein the solvent is a di-ester solvent selected from the group consisting of diethyl succinate, dibutyl succinate, diethyhexyl succinate, diisopropyl sebacate, dimethyl sebacate, diethyl sebacate, dibutyl sebacate, diisostearyl dimer, diisostearyl malate, isostearyl stearoyl stearate, isocetyl stearoyl stearate, octyldodecyl stearoyl stearate, diethylhexyl malate, diethylhexyl maleate, dipropylene glycol dibenzoate, dicapryl adipate, dicaprylyl maleate, diisopropyl dimer, diisopropyl adipate, diisobutyl adipate, diisopropyl sebacate, diisostearyl dimer, diethyhexyl succinate, diethylene glycol diethylhexanoate, neopentyl glycol dicaprate, propylene glycol dicaprylate/dicaprate, neopentyl glycol diisostearate, neopentyl glycol diethylhexanoate, neopentyl glycol diheptanoate, and combinations thereof.

19. The method of claim 14, wherein the solvent is:
(a) a citrate ester solvent of formula (X) 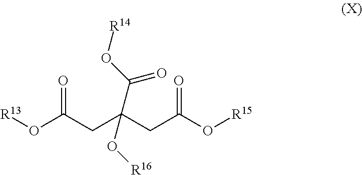
wherein
R¹³, R¹⁴, R¹⁵, and R¹⁶ are independently H, C₁-C₃₅ alkyl group, C₁-C₃₅ heteroalkyl group, C₂-C₃₅ alkene group, or C₂-C₃₅ heteroalkene group; or
(b) an ether solvent of formula (XI) 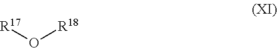
wherein
R¹⁷ and R¹⁸ are independently C₂-C₂₀ alkyl group, C₂-C₂₀ heteroalkyl group, C₂-C₂₀ alkene group, or C₂-C₂₀ heteroalkene group; or
(c) a carbonate solvent of formula (XII) 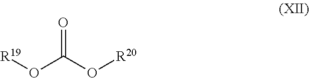
wherein
R¹⁹ and R²⁰ are independently C₂-C₂₀ alkyl group, C₂-C₂₀ heteroalkyl group, C₂-C₂₀ alkene group, or C₂-C₂₀ heteroalkene group.

20. The method of claim 14, wherein the solvent is:
(a) a citrate ester solvent selected from the group consisting of tricaprylyl citrate, triisostearyl citrate, triisocetyl citrate, trioctyldodecyl citrate, triethyl citrate, tributyl citrate, acetyl triethyl citrate, acetyl tributyl citrate, trioctyldodecyl citrate, triisocetyl citrate, and combinations thereof, or
(b) an ether solvent selected from the group consisting of dicaprylyl ether, didecyl ether, panthenyl ethyl ether, dicetyl ether, dimyristyl ether, distearyl ether, distearyl ether, dilauryl ether, and combinations thereof, or
(c) a carbonate solvent selected from the group consisting of dicaprylyl carbonate, diethyl hexyl carbonate, and combinations thereof.

21. The method of 14, wherein the solvent is a hydrocarbon with number of carbon atoms from C₄ to C₆₀.

22. The method of claim 14, wherein the solvent is:
(a) a hydrocarbon solvent selected from the group consisting of farnesene, hydrogenated farnesene, coconut alkanes, coconut/palm kernel alkanes, C₉-C₁₂ alkane, C₁₀-C₁₃ alkane, C₁₂-C₁₇ alkane, C₁₃-C₁₄ alkane, C₁₃-C₁₅ alkane, C₁₄-C₁₇ alkane, C₁₄-C₁₉ alkane, C₁₄-C₂₀ alkane, C₁₄-C₂₂ alkane, C₁₅-C₁₉ alkane, C₂₁-C₂₈ alkane, C₁₇-C₂₃ alkane, C₉-C₁₂ isoalkane, C₉-C₁₃ isoalkane, C₉-C₁₄ isoalkane, C₉-C₁₆ isoalkane, C₁₀-C₁₁ isoalkane, C₁₀-C₁₂ isoalkane, C₁₀-C₁₃ isoalkane, C₁₁-C₁₂ isoalkane, C₁₁-C₁₃ isoalkane, C₁₁-C₁₄ isoalkane, C₁₂-C₁₄ isoalkane, C₁₂-C₁₅ isoalkane, C₁₂-C₂₀ isoalkane, C₁₃-C₁₄ isoalkane, C₁₃-C₁₆ isoalkane, C₁₄-C₁₆ isoalkane, C₁₅-C₁₉ isoalkane, C₁₀-C₁₆ olefin, C₁₂-C₁₈ olefin, C₁₈-C₂₆ olefin, C₂₀ olefin, C₂₀-C₂₄ olefin, C₂₄-C₃₀ olefin, C₂₆-C₂₈ olefin, C₂₆-C₅₄ olefin, C₂₈-C₃₆ olefin, C₂₈-C₅₂ olefin, C₃₀-C₃₈ olefin, C₃₀-C₄₅ olefin, C₄-C₁₂ olefin, C₄-C₆ olefin, C₅-C₆ olefin, hydrogenated poly(C₆/C₁₀/C₁₄ olefin), hydrogenated poly(C₆-C₁₂ olefin), hydrogenated poly(C₆-C₁₄ olefin), hydrogenated poly(C₆-C₂₀ olefin), hydrogenated poly(C₈/C₁₂ olefin), poly(C₂₀-C₂₈ olefin), poly(C₃₀-C₄₅ olefin), poly(C₄-C₁₂ olefin), poly(C₆-C₁₄ olefin), hexadecene, C₃₂ alkane, C₃₂ isoalkane, C₅₄ alkane, C₅₄ isoalkane, diethylhexylcyclohexane, undecane, tridecane, tetradecane, pentadecane, hexadecane, octadecane, docosane, squalane, hydrogenated polyisobutene, polybutene, hydrogenated polydecene, hydrogenated didecene, mineral oil, liquidum, petrolatum, dodecane, isohexadecane, isododecane, isoeicosane, and combinations thereof, or
(b) a silicone solvent selected from the group consisting of dimethicone, phenyl dimethicone, caprylyl methicone, ethyl trisiloxane, cyclotetrasiloxane, cyclopentasiloxane, cyclohexasiloxane, and combinations thereof.

23. A gel or a paste prepared by contacting the elastomer of claim 1 with one or more solvents; or a powder or film prepared from the elastomer of claim 1.

24. (canceled)

25. A personal care formulation comprising the gel, paste, powder, or film of claim 23, wherein the personal care formulation is selected from the group consisting of a deodorant, an antiperspirant, a skin cream, a facial cream, a hair shampoo, a hair conditioner, a mousse, a hair styling gel, a hair spray, a protective cream, a lipstick, a facial foundation, blushes, makeup, a mascara, a skin care lotion, a moisturizer, a facial treatment, a personal cleanser, a facial cleanser, a bath oil, a perfume, a shaving cream, a pre-shave lotion, an after-shave lotion, a cologne, a sachet, a sunscreen, and combinations thereof.

26.-27. (canceled)

28. A method of preparing an elastomer comprising:
(a) reacting:
(i) at least one activated polycarboxylic acid; and
(ii) at least one polyol;
wherein the reacting of (i) and (ii) occurs in the absence of solvent in a first reaction stage, wherein the first reaction stage is for a period of at least 2 hours; and
(b) adding at least one solvent in a second reaction stage.

29. (canceled)
